(19) United States
(12) Patent Application Publication
(10) Pub. No.: US 2023/0017014 A1
(43) Pub. Date: Jan. 19, 2023

(54) BIOREACTOR WITH ENHANCED GAS TRANSFER AND THERMAL REGULATION

(71) Applicant: UNIVERCELLS TECHNOLOGIES S.A., Nivelles (BE)

(72) Inventors: Jean-Christophe Drugmand, Wavre (BE); Bart Karel Johan Heeran, Brussels (BE); Christophe Dumont, Haine-Saint-Pierre (BE); Xavier Gabet, Brussels (BE); José Castillo, Brussels (BE); Bastien Mairesse, Uccle (BE); Sebastien Jean-Pierre Michel Rodriguez, Ecaussines-Lalaing (BE); Florent Van Hijfte, Nivelles (BE)

(21) Appl. No.: 17/781,772

(22) PCT Filed: Dec. 2, 2020
(86) PCT No.: PCT/EP2020/084317

§ 371 Date: Jun. 2, 2022

Related U.S. Application Data
(60) Provisional application No. 63/004,706, filed on Apr. 3, 2020.
(60) Provisional application No. 62/942,345, filed on Dec. 2, 2019.

(51) Int. Cl. C12M 1/04; C12M 1/00; C12M 1/12

(52) U.S. Cl. CPC C12M 27/04; C12M 23/34; C12M 25/18

(57) ABSTRACT

An apparatus for culturing cells with enhanced gas transfer and thermal regulation is provided. The apparatus includes a bioreactor with a cell culture bed, such as a fixed structured bed. A pump, such as an agitator, serves to pump liquid through the cell culture bed, and a container is provided for the agitator. A first conduit may be associated with the container, such as by being connected to it or adjacent an opening into it. The bioreactor may also include flow extenders to enhance the gas transfer to a liquid used as media to culture the cells, as well as optional thermoregulators. Related methods are also disclosed.

This application claims priority to U.S. Provisional Patent Application Ser. No. 62/942,345, filed Dec. 2, 2019, and U.S. Provisional Patent Application Ser. No. 63/004,706, filed Apr. 3, 2020, the disclosures of which are incorporated herein by reference. This application further incorporates by reference U.S. Provisional Patent Application Ser. Nos. 62/758,152, 62/733,375, and 62/608,261; U.S. Patent Application Publication No. 2018/0282678; International Patent Application PCT/EP2018/076354; U.S. Provisional Patent Application 62/711,070; and U.S. Provisional Patent Application 62/725,545.

### TECHNICAL FIELD

This document relates generally to the cell culturing arts and, more particularly, to a bioreactor with enhanced gas transfer and thermal regulation.

### BACKGROUND

Bioreactors are frequently used for culturing cells. Within the bioreactor, a certain level of gas transfer between the gas phase (e.g. air or oxygen) and the liquid phase (the cell culture medium) is required to permit optimal cell growth. A supply of oxygen to the cells is mandatory in such gas transfer.

Gas transfer is especially important for a fixed-bed bioreactor, the high cell density growth of which demands a heightened level of oxygen. The amount of oxygen present in the cell culture medium for enhancing cell growth may be assessed using the concept of gas transfer coefficient, or kLa. This value uses the volumetric mass-transfer coefficient that describes the efficiency with which gas can be delivered to the cell culture medium in a bioreactor for a given set of operating conditions and surfaces.

In the past, proposals have been made for enhancing the gas transfer by providing the bioreactor with one or more bubblers or spargers to form small gas bubbles in the cell culture medium. However, when a sparger is used to support a high cell density bioreactor, the speed of bubble input and the need for an extensive amount of bubbles often results in the negative effects of shear and excessive foaming. This can deleteriously cause undue mechanical and oxidative stress to the cells, which can impact yields. Further, foaming can clog filters and other connectors in the bioreactor. In addition, use of spargers in unstructured high cell density fixed bed bioreactors is challenging as the resulting bubbles can cause large “clouds” or of air pockets as the bubbles have no definable path through the fixed bed. Such bubbles negatively affect homogeneity of the bioreactor conditions and, thus, cell growth.

Likewise, certain fixed bed bioreactors provide for liquid-to-gas contact, such as in a headspace above the fixed bed. While current implementations do serve to provide for gas transfer to the circulating liquid media, the residence time of this liquid in contact with the gas has been limited. Gas transfer would be enhanced by increasing such residence time. It would be further enhanced by also creating turbulence in the liquid while in contact with the gas phase.

It may also be desirable to regulate (typically increase) the internal temperature of a bioreactor. In the past, this has been achieved using an external heater, such as a film applied to an outer surface of the bioreactor. As can be appreciated, this approach is inefficient, given that the heat must transfer through the external wall of the bioreactor, which typically has a high thermal resistance (and even if not, the transferred heat may only directly influence a portion of any contained liquid media proximate to the wall interior). Poor contact between the film and the outer wall can also lead to significant losses. Hence, using the current external heating approach, it can take a significant amount of time to cause even a modest temperature increase, and can be challenging to precisely regulate the temperature of the liquid throughout the bioreactor. Internal heating can be challenging for several reasons including cost and complexity. Also, heat can impair cell growth within the bioreactor if directly applied to bed where cells reside.

Accordingly, a need is identified for a bioreactor, particularly a high cell density fixed bed bioreactor, with enhanced gas transfer, and further with optional thermal regulation capabilities.

### SUMMARY

According to a first aspect of the disclosure, an apparatus for culturing cells comprises a bioreactor including a fixed cell culture bed. An agitator is provided for pumping liquid through the cell culture bed. A container is provided for the agitator, and a first conduit connected to the container.

In one embodiment, the first conduit comprises an injector conduit for delivering gas bubbles into the container. In another embodiment, the first conduit comprises a drain conduit for draining liquid from the bioreactor. In the former case, the apparatus may further include a second conduit comprising a drain conduit for draining liquid from the bioreactor.

In one embodiment, the bioreactor comprises a central chamber and a cover. The first conduit extends from the cover and through the central chamber to an opening of the container. The apparatus may further include a tubular post associated with the container, the first conduit being connected to the tubular post.

The agitator may comprise a, preferably magnetic, impeller rotatably coupled to the tubular post by a bearing. The agitator may include a plurality of curved vanes. Additionally, or alternatively, the agitator comprises a plurality of vanes, each vane of the plurality of vanes having a higher central portion and a lower peripheral portion so as to create a variable height.

The container may comprise a sloped upper portion adapted to accommodate the variable height agitator. The container includes a fitment adapted to form custom-sized bubbles from a gas delivered thereto by the first conduit. The fitment comprises a perforated portion.

The cell culture bed may be adapted to divide/shear any gas bubbles in the liquid. The cell culture bed may comprise a fixed bed, such as a structured fixed bed. The structured fixed bed may comprise alternating layers of one or more cell immobilization layers, and optionally one or more spacer layers. The one or more cell immobilization layers or the one or more spacer layers form paths to divide or shear gas bubbles in the liquid that travel through the fixed bed.

The cell culture bed may be located in an annular outer chamber of the bioreactor adapted to receive a flow of liquid from a pumping action created by rotation of the agitator in the container, or may be positioned in a container having any other shape (such as a container with a square or rectangular cross-section and arranged to allow liquid to flow through the fixed bed from side to side). The apparatus may further include a plurality of cell culture beds arranged in a stacked configuration within an annular outer chamber of the bioreactor.

In any of the foregoing embodiments, the bioreactor may further include a flow extender, possibly with a thermoregulator, as disclosed herein.

According to a further aspect of the disclosure, an apparatus for culturing cells, comprises a bioreactor including a cell culture bed. An agitator is provided for pumping liquid through the cell culture bed. An injector is provided for injecting a gas into the liquid to form first bubbles having a first size, the agitator converting the first bubbles to second bubbles having a second, smaller size than the first size for delivery to the cell culture bed with the liquid.

The agitator is located in a container in fluid communication with the bioreactor, the container including at least one inlet and at least one outlet. The injector may comprise a tube having an open end located upstream of the at least one inlet to the container. A housing having a central chamber associated with the at least one inlet may be located radially inward of the cell culture bed. An open end of the tube may be positioned within the central chamber. The injector may comprise a tube having an open end located in a fluid flow path between the at least one inlet and the at least one outlet.

The cell culture bed may comprise a fixed bed, such as a structured fixed bed. The structured fixed bed may comprise alternating layers comprising one or more cell immobilization layers and one or more spacer layers. A channel may be formed between the one or more cell immobilization layers and one or more spacer layers to divide/shear any gas bubbles in the liquid into a third bubbles having a third yet smaller size than the second size.

In accordance with a further aspect of the disclosure, an apparatus for culturing cells includes a bioreactor including a cell culture bed, means for pumping liquid through the cell culture bed, and means for injecting a gas into the liquid to form first bubbles having a first size, the pumping means converting the first bubbles to second bubbles having a second, smaller size than the first size for delivery to the bed with the liquid. The means for pumping may comprise an agitator. The means for injecting the gas may comprise a tube connected to a gas supply.

Still a further aspect of the disclosure pertains to a method of culturing cells in a cell culture bed. The method comprises providing a liquid with gas bubbles, agitating the liquid to divide the gas bubbles, and delivering the liquid with the divided gas bubbles to the cell culture bed. The providing step may comprise supplying a gas to the liquid upstream of an agitator for performing the agitating step, or supplying a gas to the liquid to a container including an agitator for performing the agitating step.

In one embodiment, the the cell culture bed comprises alternating layers comprising one or more cell immobilization layers and, optionally, one or more spacer layers forming a channel for the liquid through the bed. One or more of the layers perform the step of further dividing the bubbles as the liquid passes through the cell culture bed.

Yet a further aspect of the disclosure pertains to a method for culturing cells. The method comprises a bioreactor including a cell culture bed. An agitator is provided for pumping liquid through the cell culture bed. An injector is also provided for injecting a gas into the liquid to form bubbles having a first size. The agitator is adapted to divide or shear the gas bubbles into a second size smaller than the first size, and the cell culture bed is adapted to divide or shear the gas bubbles into a third size smaller than the second size.

Still a further aspect of the disclosure relates to an apparatus for culturing cells. The apparatus comprises the steps of providing a liquid with gas bubbles having a first size, agitating the liquid to divide the gas bubbles to a second size smaller than the first size, and delivering the liquid with the divided gas bubbles to the cell culture bed, which divides the bubbles to a third size smaller than the second size.

A further aspect of the disclosure pertains to an apparatus for culturing cells comprising a housing for containing a liquid for delivery to a bed for culturing cells, the housing including a zone having a gas. A flow extender is adapted for extending a residence time and causing turbulence in the liquid in the zone.

In one embodiment, the flow extender comprises a frusto-conical structure having surface structures for causing the turbulence. The flow extender may comprise one or more labyrinthian, maze-like or winding passages. The flow extender may comprise a plurality of steps.

The housing may be arranged with an outer chamber for receiving the bed, the chamber being an upper chamber including a headspace being located above a liquid exit of the bed, and further including a central chamber for returning liquid for entering the bed. The housing may be arranged with an outer chamber for receiving the bed, the chamber being a central chamber for returning liquid for entering the bed. The housing may be arranged with an inner chamber for receiving the bed, the chamber being an outer chamber for returning liquid for entering the bed.

In one embodiment, the flow extender has a peripheral edge greater in height relative to a radially inward edge of the extender. The housing may comprise an annular chamber including the bed and having a radial dimension X, and wherein a radial dimension Y of the extender is greater than 0.5X. The flow extender may be capable of being heated.

Still further, an apparatus for culturing cells is provided. The apparatus comprises a housing for containing a liquid for delivery to a bed for culturing cells, the housing including a headspace having a gas. A flow extender is adapted to extend a residence time in the headspace. The flow extender may comprise a frusto-conical plate and/or one or more maze-like or winding passages. The flow extender may comprise a plurality of steps.

In one embodiment, the housing is arranged with an outer chamber for receiving the bed, the headspace being located above a liquid exit of the bed, and further including a central chamber for returning liquid for entering the bed. The flow extender may have a peripheral edge greater in height relative to a radially inward edge of the extender. The housing may comprise an annular chamber including the bed and having a radial dimension X, and wherein a radial dimension Y of the extender is greater than 0.5X. In any disclosed embodiment, the flow extender, if present, may be heated.

This disclosure also relates to an apparatus for culturing cells including a housing for containing a liquid for delivery to a bed for culturing cells, the housing including a centrifugal pump for pumping liquid through the bed and into a headspace. A flow extender is adapted for increasing a residence time of the liquid in the headspace. An injector is provided for injecting a gas to the liquid to form first bubbles having a first size, the centrifugal pump converting the first bubbles to second bubbles having a second, smaller size than the first size for delivery to the bed with the liquid being pumped.

Still another portion of this disclosure relates to an apparatus for culturing cells comprising a housing for containing a liquid for delivery to a bed for culturing cells. A thermoregulator is located within the housing for regulating a temperature of the liquid without directly heating or cooling the bed.

In one embodiment, the thermoregulator comprises a stepped structure. The apparatus may further include a structure having upstanding walls thermally regulated by the thermoregulator. The thermoregulator may be located in a headspace of the housing. The thermoregulator may include a power source external to the housing and a resistive element internal to the housing. The thermoregulator may comprise a radiator.

Still another part of the disclosure relates to an apparatus for culturing cells. The apparatus comprises a housing for containing a liquid for delivery to a bed for culturing cells, the housing including a headspace having a gas. A flow extender is adapted for extending a residence time and/or creating turbulence in the liquid in the headspace. In one embodiment, the flow extender comprises a surface having a slope of 1-85° relative to a horizontal plane.

Yet another portion of the disclosure relates to an apparatus for culturing cells. The apparatus comprises a housing for containing a liquid for delivery to a bed for culturing cells, the housing including a zone having a gas. A flow extender is adapted for extending a residence time of the liquid in the zone wherein the flow extender comprises a surface having a slope of 1-85° relative to a horizontal plane. In one embodiment, the zone comprises a headspace.

This disclosure also relates to an apparatus for culturing cells. The apparatus comprises a housing for containing a liquid for delivery to a bed for culturing cells, the housing including a zone having a gas. A flow extender is adapted for extending a residence time and/or causing turbulence in the liquid in the zone, the flow extender comprising an open shell.

In one embodiment, the open shell comprises a chamber for receiving a portion of the liquid. A regulator may be provided for regulating flow into the chamber. The open shell may comprise a sloped upper surface. A thermoregulator may be connected to the open shell.

### BRIEF DESCRIPTION OF THE DRAWING FIGURES

FIG. 1 is a perspective view of an exemplary bioreactor for which certain aspects of this disclosure may have applicability;

FIG. 2 is a partially exploded view of further details of the bioreactor of FIG. 1;

FIG. 3 illustrates a spiral fixed bed for possible use in connection with a bioreactor;

FIGS. 3A, 3B, and 3C illustrate particular details of the structured fixed bed;

FIGS. 3D, 3E and 3F illustrate alternative arrangements for forming a structured fixed bed;

FIG. 4 illustrates a container for a fluid agitator including a gas supply tube according to one aspect of the disclosure;

FIG. 5 illustrates an alternative embodiment of the container;

FIG. 5A illustrates a further embodiment of a bioreactor with a gas supply tube;

FIGS. 5B and 5C are alternative embodiments of the bioreactor of FIG. 5A;

FIG. 6 is an alternative embodiment of a bioreactor;

FIGS. 6A and 6B illustrate further details of the FIG. 6 embodiment;

FIGS. 6C and 6D illustrate details of an impeller for use in any disclosed embodiment;

FIG. 7 is a chart providing an indication of an improvement in HA values resulting from the use of a gas supply tube in the manner proposed;

FIG. 8 is a schematic diagram of an example of a flow extender for enhancing gas transfer according to one embodiment;

FIG. 9 is a schematic diagram of an example of a flow extender for enhancing gas transfer according to another embodiment;

FIG. 10 is a schematic diagram of an example of a flow extender for enhancing gas transfer according to one embodiment, and optionally including a temperature regulator, such as for example a heater;

FIG. 11 is a further illustration of the flow extender;

FIGS. 12, 12A, and 12B illustrate further embodiments of flow extenders;

FIG. 13 illustrates still a further embodiment of a flow extender;

FIG. 14 is a schematic illustration of another embodiment of a flow extender;

FIGS. 15-19 illustration further versions of flow extenders;

FIG. 19A illustrates a flow extender with turbulence generating structures;

FIGS. 20 and 21 schematically illustrate alternate forms of bioreactors with gas supply tubes;

FIG. 23 a chart providing an indication of an improvement in kLA values resulting from the use of a flow extender in the manner proposed;

FIGS. 22, 22A and 22B illustrate a possible environment of use of a bioreactor according to the disclosure;

FIGS. 24 and 25 illustrate alternative embodiments of flow extenders with thermoregulation capabilities;

FIGS. 26-27 illustrate further embodiments.

### DETAILED DESCRIPTION

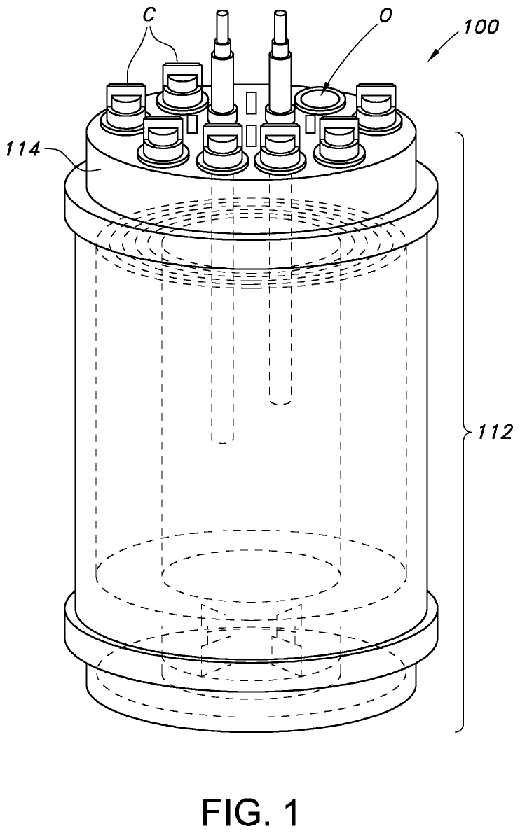

Reference is now made to FIGS. 1-3, which illustrate one embodiment of a fixed bed bioreactor 100 for culturing cells, according to one aspect of the disclosure. In some embodiments, the bioreactor 100 includes an external casing or housing 112 forming or including an interior compartment and a cover 114 placed on top of the housing 112 to cover or seal the interior compartment after it is populated with at least the fixed bed. In an embodiment, the cover 114 is removable. The cover 114 may include various openings or ports O with removable closures or caps C for allowing for the selective introduction or removal of material, fluid, gas, probes, sensors, samplers, or the like.

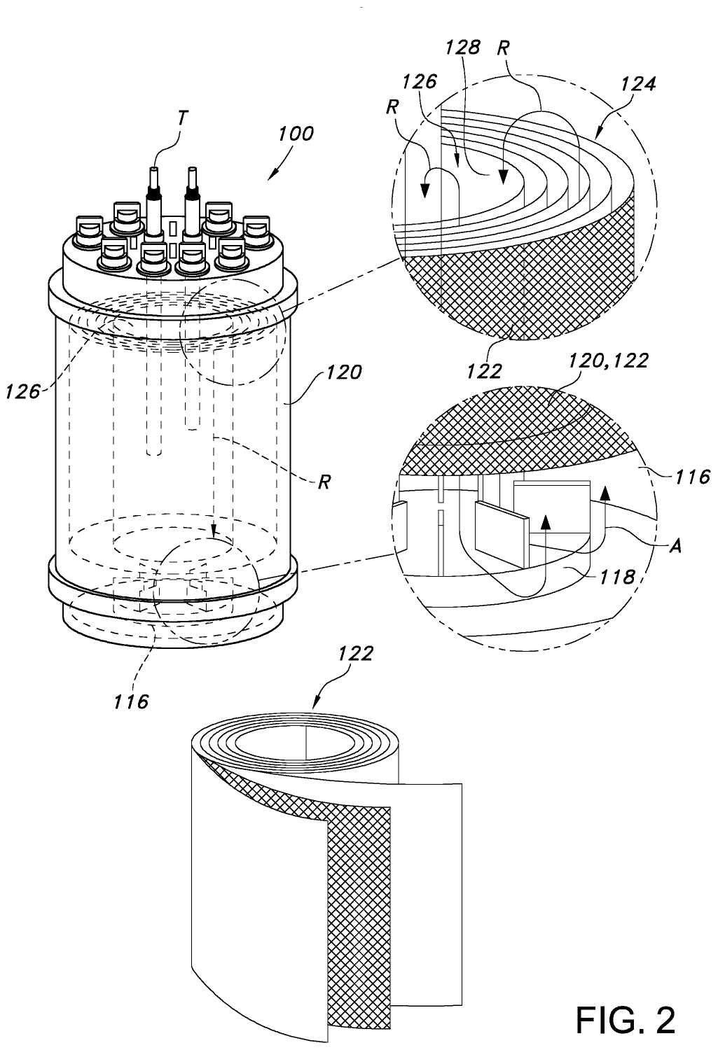

Within the interior compartment of the bioreactor housing 112, several compartments or chambers may be provided for transmitting a flow of liquid, gas, or both, throughout the bioreactor 100. As indicated in FIG. 2, in some embodiments, the chambers may include a first chamber 116 at or near a base of the bioreactor 100. In some embodiments, the first chamber 116 may include an agitator for causing liquid flow within the bioreactor 100. The agitator may be in the form of a rotatable, non-contact magnetic impeller 118, which thus forms a centrifugal pump in the bioreactor. The agitator could also be in the form of an impeller with a mechanical coupling to the base (e.g., via a bearing), with a contact or non-contact drive, or perhaps even an external pump forming part of a liquid circulation system, or any other device for causing liquid circulation within the bioreactor.

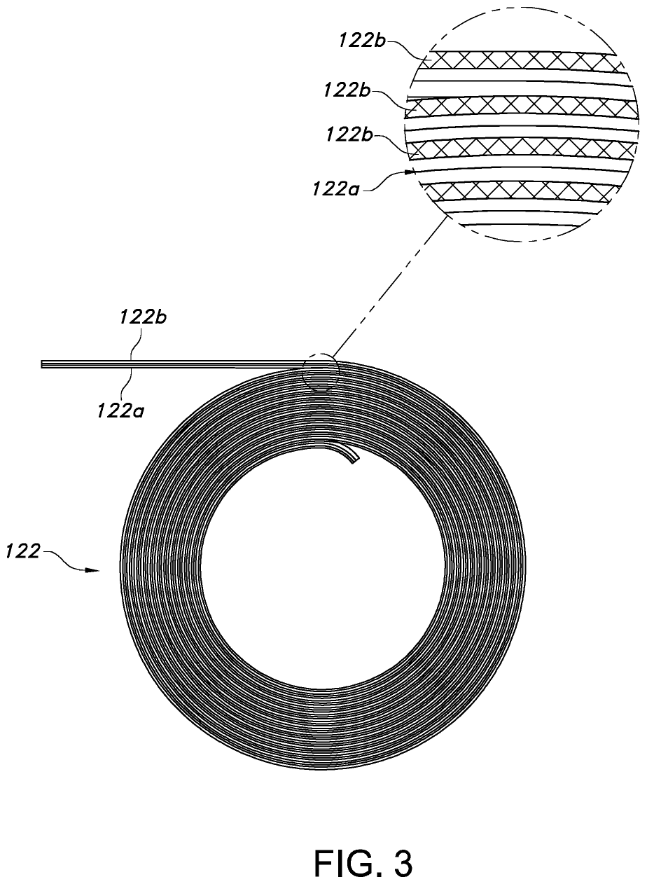

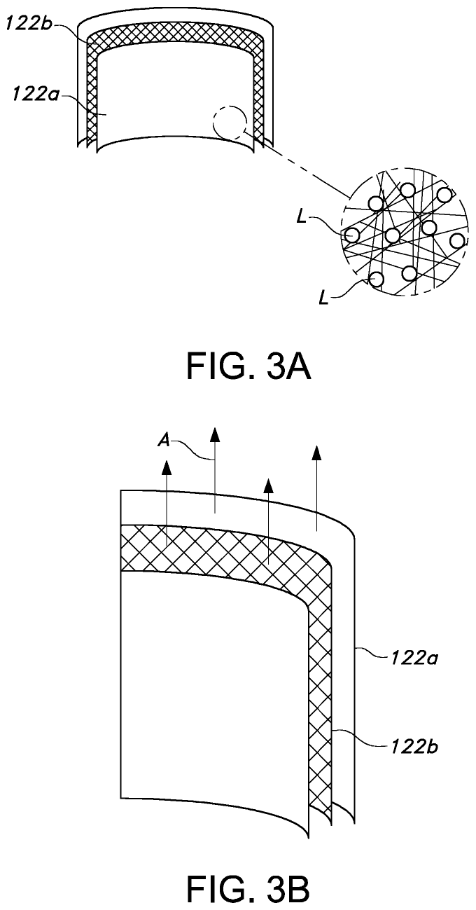

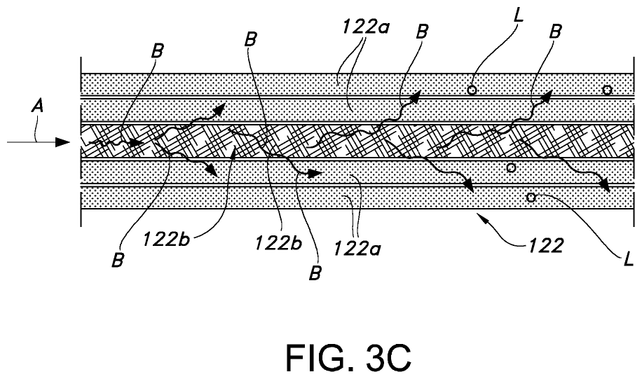

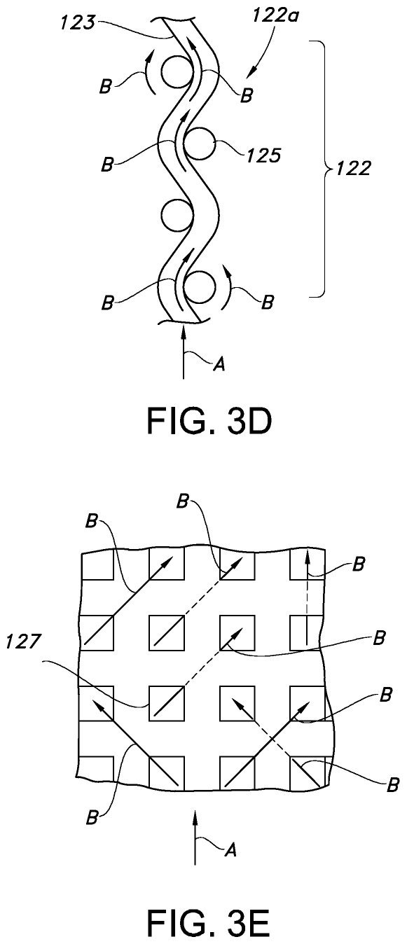

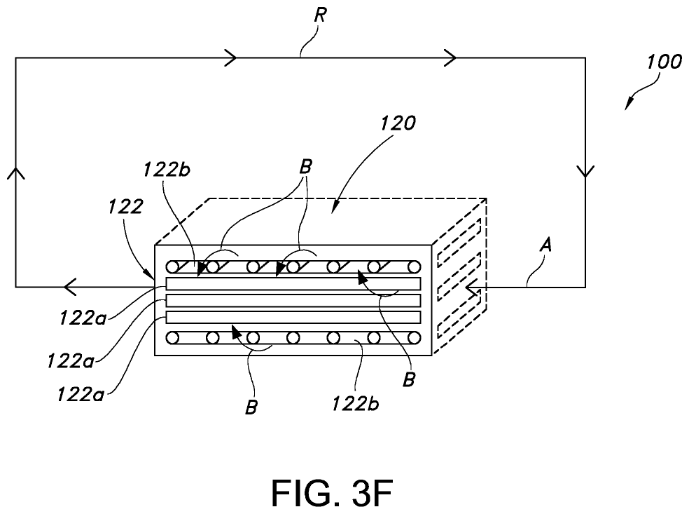

As a result of the agitation and pumping action provided by the agitator (impeller 118), liquid may then flow upwardly (as indicated by arrows A in FIG. 2) into a chamber 120 along the outer or peripheral portion of the bioreactor 100 (or otherwise through the fixed bed). In some embodiments, the bioreactor is adapted to house a cell culture bed in any form including a packed bed, fixed bed, structured fixed bed, fluidized bed, etc. FIG. 3 shows a fixed bed in the form of a structured spiral bed 122 which, in use, may contain and retain cells being grown. In some embodiments, the spiral bed 122 may be in the form of a cartridge that may be built within and as a part of or introduced into the outer chamber 120. The bed 122 can be pre-installed in the chamber during manufacture at a facility prior to shipping or installed at the point of use.

Liquid exiting the chamber 120 is passed to a “headspace” formed in a chamber 124 between one (upper) side of the bed 122 and the cover 114, where the liquid (media) is exposed to a gas (such as oxygen). In some embodiments, liquid may then flow radially inwardly to a central chamber 126 to return to the lower portion of bed 122. In some embodiments, this central chamber 126 can be columnar in nature and may be formed by an imperforate conduit or tube 128 or rather formed by the central opening of the structured spiral bed.

The chamber 126 returns the liquid to the first chamber 116 (return arrow R) for recirculation through the bioreactor 100, such that a continuous loop results (“bottom to top” in this version). In some embodiments, a sensor, for example a temperature probe or sensor T may also be provided for sensing the temperature of the liquid flowing or residing in the chamber 126. In some embodiments, additional sensors (such as, for example, pH, oxygen, dissolved oxygen, temperature, cell density, etc.) may also be provided at a location before the liquid enters (or re-enters) the chamber 116, including for example at the exit location, or top, of the fixed bed 122.

FIG. 3 shows one embodiment of a matrix material for use as a structured fixed bed 122 in the bioreactor of the present disclosure and, in particular, a spiral bed. In some embodiments, one or more cell immobilization layers 122a are provided adjacent to one or more spacer layers 122b made from a mesh structure. In some embodiments, the layering may optionally be repeated several times to achieve a stacked or layered configuration.

In some embodiments, the mesh structure included in spacer layers 122b forms tortuous paths to steer the cells into the depth of the cell immobilization layers 122a (see cells L in FIG. 3A suspended or entrapped in the material of the immobilization layer 122a). As shown in FIGS. 3B and 3C, the spacer layers 122b form tortuous channels in conjunction with the adjacent cell immobilization layers 122a for liquid and bubbles to flow therethrough. Arrows A in FIGS. 3B and 3C illustrate a primary direction of flow of the liquid and bubbles through the fixed bed between layers and arrows B in FIG. 3C illustrate accompanying secondary variable and at least partially transverse flow directions of the liquid and bubbles due to the geometry of the spacer layer resulting in cells penetrating into various depths of the adjacent cell immobilization layers. Critically, increased homogeneity of the cells is maintained within the structured fixed bed as a result of this type of arrangement.

As shown in FIGS. 3, 3A and 3B, the structured fixed bed can be spirally or concentrically rolled along an axis or core (e.g., conduit or tube 128, which may be provided in multiple component parts). In some embodiments, the layers of the structured fixed bed are firmly wound. In some embodiments, the diameter of the core, the length and/or amount of the layers will ultimately define the size of the assembly or matrix. In some embodiments, thickness of each of the layers 122a, 122b may be between 0.1 and 5 mm, 0.1 and 10 mm, or 0.001 and 15 mm.

In some embodiments, other structures can be used which form such tortuous paths. For example, FIG. 3D shows that the one or more cell immobilization layers 122a may be adapted to form a structured fixed bed 122. The one or more layers 122a provide a tortuous channel of flow (arrow B) from a linear or regular inflow (arrow A) without using additional spacer layers (but such may be used, if desired). This may be achieved, for example, by providing a layer of woven fibers or filaments 123, 125 that serve to disrupt the flow, which may serve to further shear or divide any bubbles present and thus enhance kLa values in the fixed bed 122.

FIG. 3E shows that such a result may be achieved using a non-woven material as the cell immobilization layer 122a. This may be achieved by forming the layer 122a as a reticulated arrangement (such as by 3-D printing) with openings 127 through which liquid may pass and return again, thus forming the tortuous channels that again promote homogeneity and also serve to further shear or divide any bubbles present in the liquid. This function may again be achieved with or without added spacer layers being present.

The orientation of the structured fixed bed 122 may be other than as shown in a bioreactor 100 as shown in FIG. 2, where the flow is arranged vertically (bottom to top, in the example provided). For example, as shown in FIG. 3F, a bioreactor 100 may include a first chamber 120 that includes a structured fixed bed 122 comprised of one or more horizontally arranged material layers. The one or more layers may comprise a woven or reticulated material, as per FIGS. 3D and 3E, but as illustrated in FIG. 3F, may comprise one or more cell immobilization layers 122a (three shown, but any number may be present) sandwiched by adjacent spacer layers 122b (vertical spacing exaggerated for purposes of illustration). The flow is thus arranged from side-to-side (left to right or right to left), with the material layer(s) (spacer or otherwise) providing for the channels for creating the tortuous flow (arrows B) from a linear or regular inflow (arrow A) and thus serving to further divide any bubbles present in the liquid. The pumping action may be provided by an agitator or other pump at the entrance end of the chamber 120, and a return path provided at the exit end, as schematically illustrated by path R. Additional spacer layers may also be provided between the cell immobilization layers 122a, if desired.

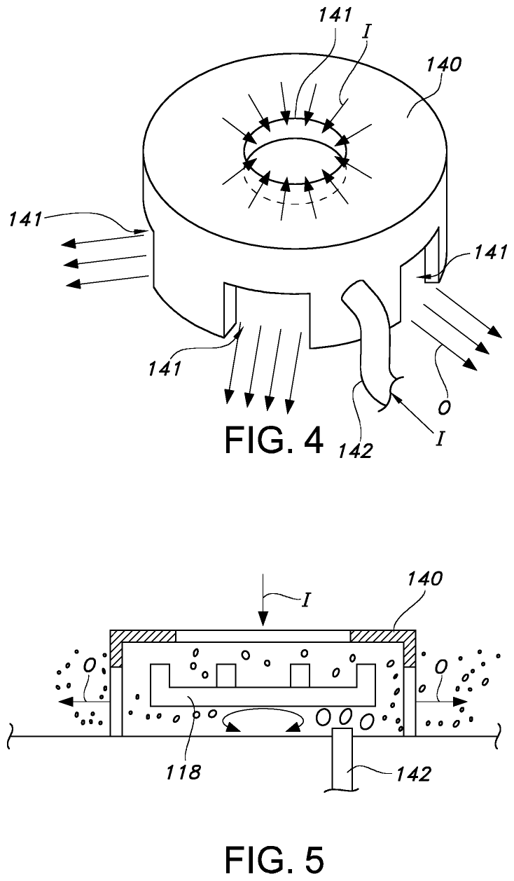

As noted above and more evidently indicated in FIG. 4, the chamber 116 may be formed of an agitator housing or container 140. One skilled in the art will appreciate that the walls such container 140 may be formed independent of the rest of the bioreactor vessel or may be shared with other walls or parts of the bioreactor vessel. For instance, in FIG. 4, the bottom of such container is formed by the bottom of the bioreactor vessel. To allow for liquid flow, the container 140 may include a plurality of openings 141, one or more of which may serve as either inlets or outlets for admitting and releasing liquid (note exemplary action arrows I (IN) and O (OUT) in FIG. 4). In some embodiments, liquid exiting the container 140 may then flow upwardly (as indicated by arrows in FIG. 2) and through the structured bed, such as spiral bed 122, and return to an inlet via openings 141 of the container 140 via the central chamber 126. One skilled in the art will appreciate that the arrangement could also operate in reverse, as outlined further in the following description.

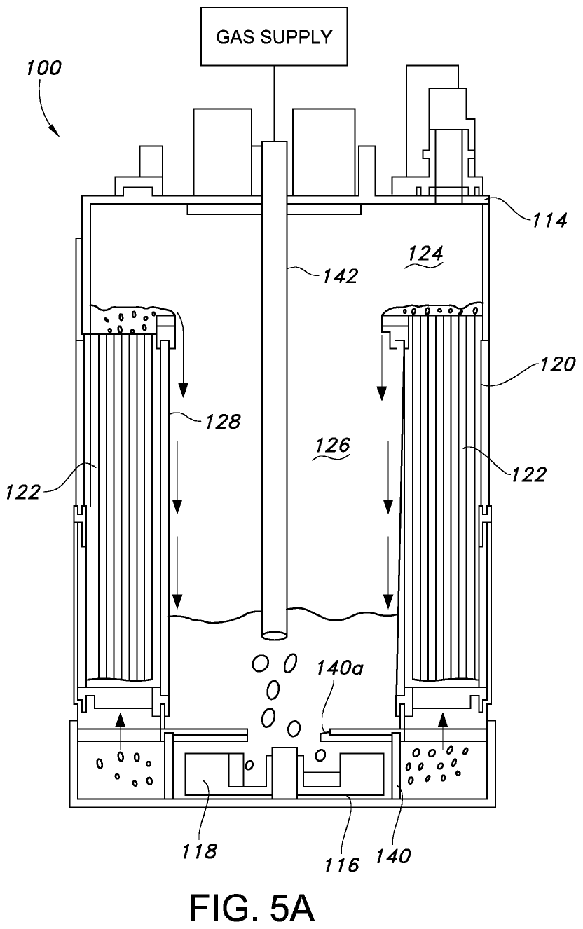

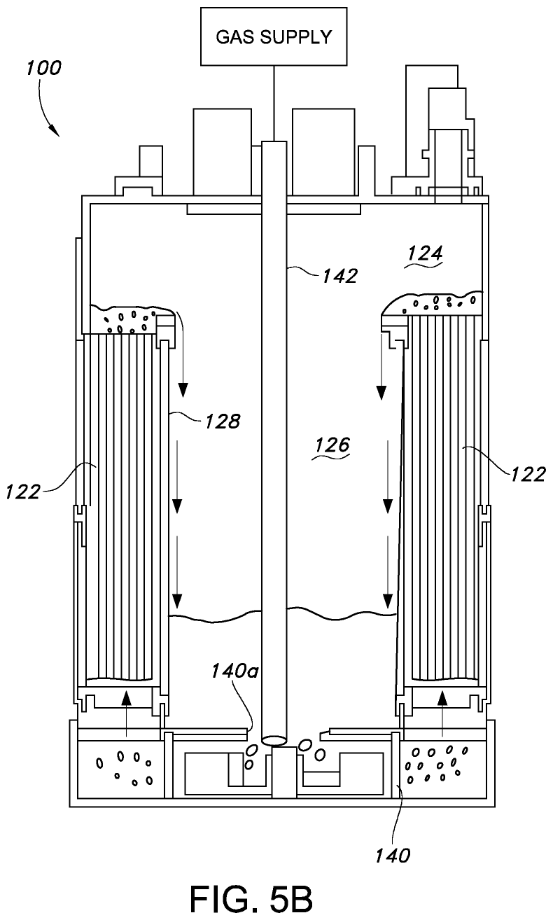

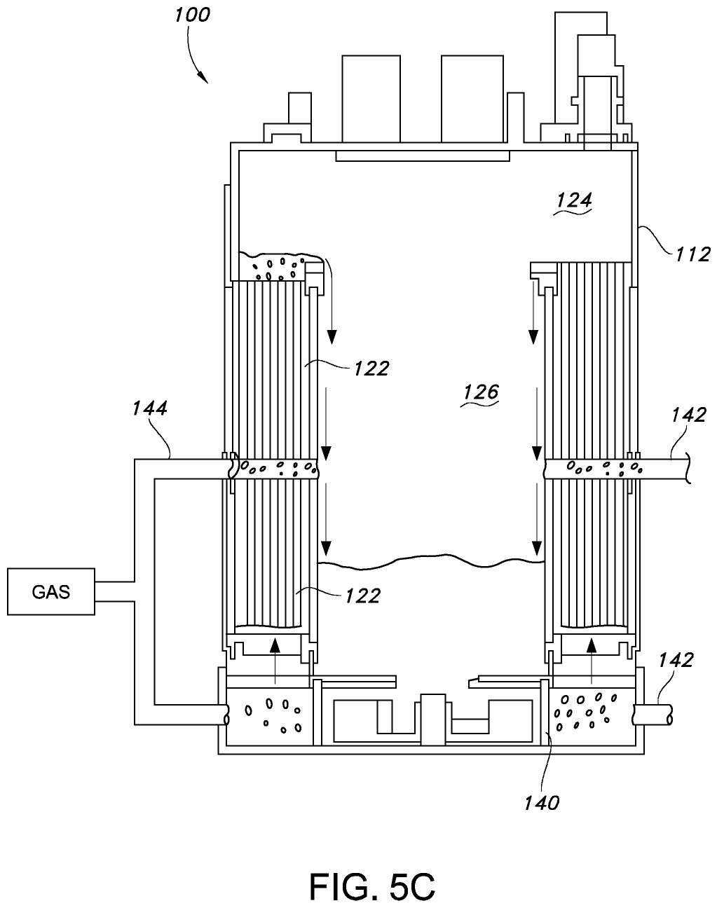

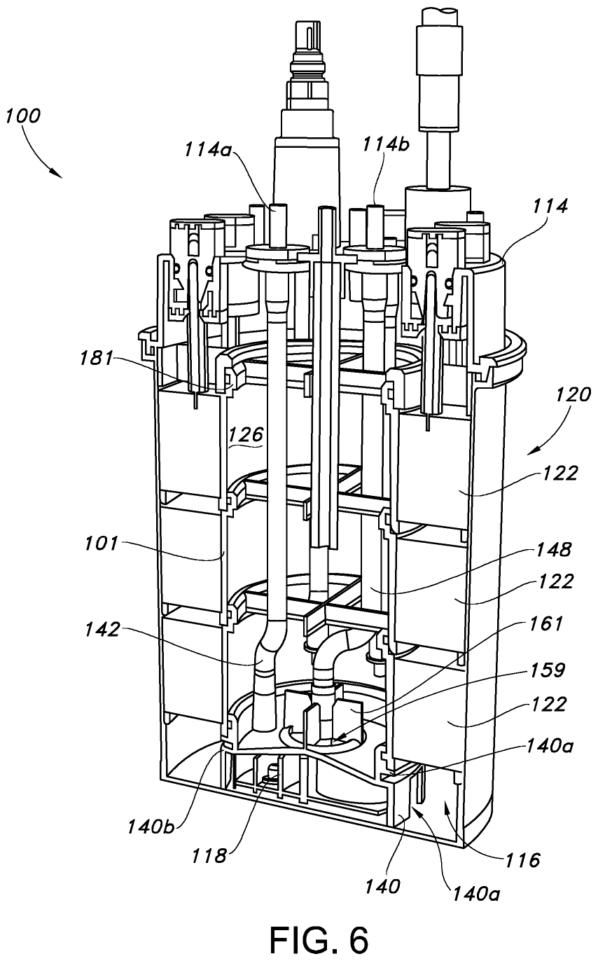

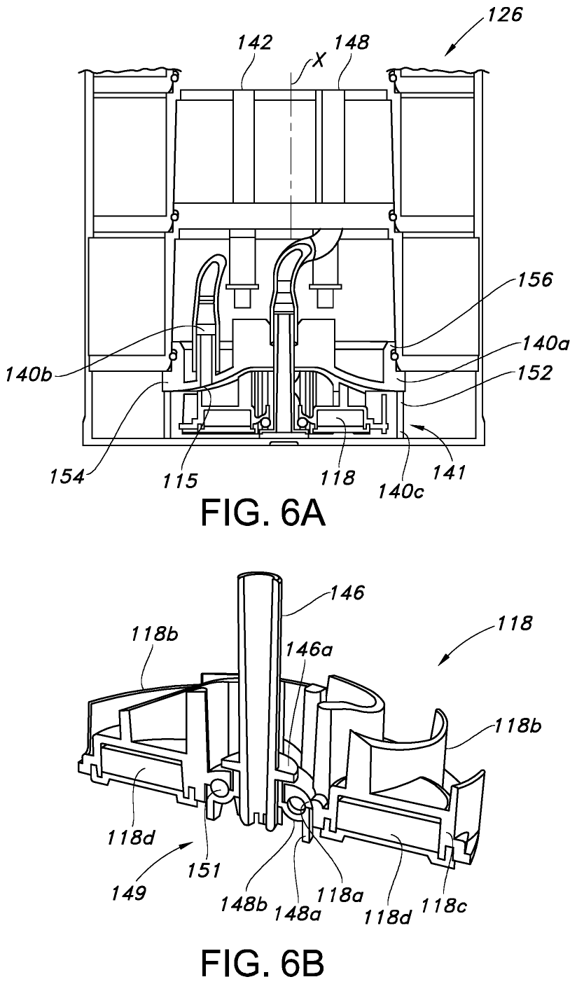

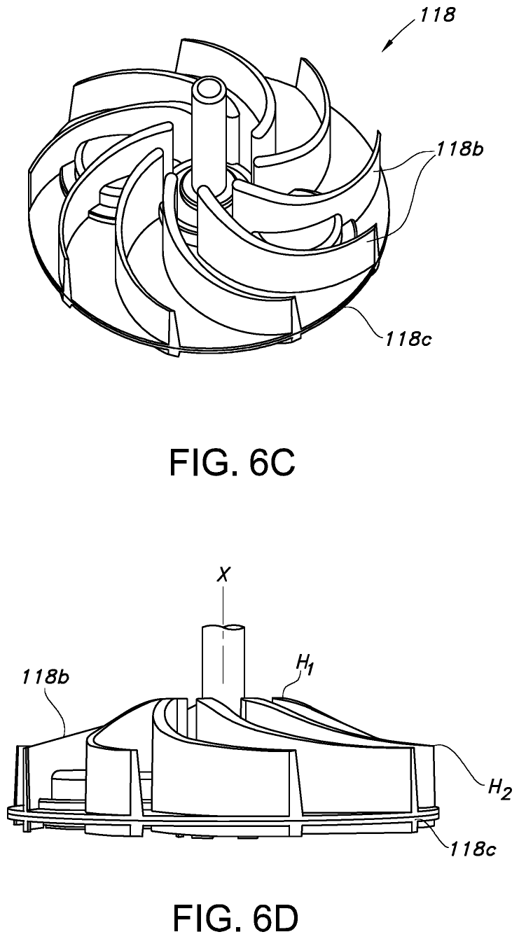

As noted previously, it may be desirable to increase the amount of gas transfer to the cell culture media liquid while circulating through the bioreactor 100. According to one aspect of the disclosure, and with reference to FIG. 5, this may be achieved by introducing a flow of gas, such as air or oxygen, into the bioreactor 100. Specifically, an injector for injecting a gas (such as sterilized air) may be provided so that bubbles are injected into the fluid at or near the centrifugal pump created by an agitator, such as impeller 118 within the container 140. In some embodiments, the injector may comprise an injector conduit or tube 142 which can be connected to a gas supply external to the bioreactor 100 (see, e.g., FIGS. 5A, 5B), and connected to any wall thereof. For instance, such tube 142 can be connected to the cover 114 of the bioreactor 100 as shown in FIGS. 5A and 5B. Alternatively, tube 142 can be connected to the bioreactor 100 at a location along the base of the bioreactor adjacent to the location of the container 140, as shown in FIG. 5. In yet another manner. Additionally, in order to ensure stability and proper injection location of bubbles, tube 142 can be connected along a sidewall of container 140, as shown in FIG. 4, or along an upper wall of the container 140, as shown in FIG. 6).

In some embodiments, as shown in FIG. 5, the outlet of the tube 142 for supplying gas to the liquid of the bioreactor 100 is within or in fluid communication with an interior of the container 140, and thus located in the fluid path between the inlet I and outlet O. However, it is also possible for the outlet of the tube 142 to be located within the central chamber 126, such as when the tube 142 is passed through another wall or surface of the bioreactor 100. In FIG. 5A, the tube 142 is shown to be passing through the cover 114 into the lower portion of the central chamber of the bioreactor proximate the container 140, and in FIG. 5B, the tube 142 is shown entering the container 140, such that an open distal end of the tube may transmit liquid directly to, or is in fluid communication directly with, the interior compartment of the container.

In any of the embodiments, oxygen-containing gas (such as air) injected into the bioreactor 100 via tube 142 forms relatively large size of bubbles in the fluid since the tube simply includes an unobstructed open end. Alternatively, smaller bubbles can be generated with the use of a device such as a sparger is used along with customized nozzles and related oscillator systems. In either case, as a result of the positioning of the open end or nozzle of the tube 142, these larger “macrobubbles” or smaller “microbubbles” (Size 1) are released to a location before which they will encounter the turbulence and shearing action created by the agitator (e.g., rotating impeller 118 serving as agitator) forming the pump for the bioreactor 100. This agitation serves to divide the larger macrobubbles into more numerous bubbles having a smaller size, or “microbubbles” or to divide the smaller microbubbles into yet more numerous bubbles having yet smaller size (Size 2). The formation of more numerous bubbles may further result from the increased residence time of the gas bubbles within the agitator container 140 (if present) as well as the speed and design of the agitator.

As a result of the rotation of impeller 118 to create the flow pattern for the bioreactor 100 shown in FIGS. 1, 1A, and 1B, these microbubbles may then be carried away by the flowing liquid, such as radially outwardly and upwardly in the illustrated bioreactor 100. It can be appreciated by one skilled in the art that the opposite flow pattern would result if the pump direction reversed.

Given their smaller size, the microbubbles are better able to pass into and through channels formed by the spacer layers 122b and the adjacent cell immobilization layers 122a (or other available paths) of the fixed bed 122. This serves to further enhance the oxygenation of the cells being grown in the bed, without a corresponding need to increase the speed of the impeller 118 and the resulting liquid flow rate. Moreover, the release of the gas into or near the agitator container 140 and the resulting flow avoids the creation of deleterious air pockets in the bioreactor 100, which are notoriously difficult to remove without halting the bioreactor operation.

However, in another embodiment of the disclosure, as the (Size 2) microbubbles pass through the structured fixed bed 122, they are further divided into smaller and more numerous microbubbles (Size 3). For example, this may occur as the bubbles enter into the channels formed in the fixed bed 122, such by the spacer layers 122b and/or the adjacent cell immobilization layers 122a and pass through all available tortuous (or labyrinthian) paths provided. As they travel, the larger bubbles tend to break up or divide into still yet smaller microbubbles before exiting the fixed bed 122. Consequently, the gas transfer during residence time in the fixed bed 122 is increased and the bubbles emanating from the bed once having passed therethrough are actually still even smaller in size (and thus more numerous) that at a point of entry, as a result of shearing forces caused by the bubbles engaging the mesh of the spacer layer 122b in the illustrated embodiment.

As indicated in FIGS. 5A and 5B, in the illustrated version of a bioreactor 122, liquid exiting from the bed 122 travels to the central chamber 126 where it flows directly along the inner surface of the wall thereof, which may be formed by tube 128. A thin layer or “film” of liquid may be created, which flows downwardly (in this embodiment) while passing through the central chamber 126. In some embodiments, this gas-liquid interface serves to increase the volume of the liquid exposed to gas (e.g., air), prior to it being returned to the first chamber 116 and eventually re-entering the fixed bed 122 as a result of the pumping action created by agitator. In some embodiments, this can allow for more oxygen transfer which may be needed for larger sizes of bioreactor or otherwise to increase cell growth rates or adjust process parameters based on the biologic being produced.

FIG. 5C illustrates a further example of a bioreactor 100, which includes multiple stacked beds (two shown, but any number could be provided). In this arrangement, the gas injector is arranged to provide a fresh supply of gas to a location at the inlet end of each bed 122 in the stack, which may be achieved using individual tubes 142 passed through a corresponding portion (e.g., housing 112a) or a manifold 144 connected to a gas supply. The bubbles may be larger in size upon introduction, and then once having passed through the corresponding bed 122 are actually still even smaller in size that at a point of entry, as a result of shearing forces caused by the bubbles engaging the mesh of the spacer layer 122b in the illustrated embodiment. Alternatively, the bubbles may be smaller due to use of a sparger/nozzle system perhaps with an oscillator so that they do not create a cloud an inlet of fixed bed section.

A further embodiment of a bioreactor 100 is shown in FIGS. 6, 6A, and 6B, which again includes multiple stacked fixed beds 122 (three shown, but any number could be provided). As in prior embodiments, the bioreactor 100 in this embodiment includes a cover 114 in an upper portion. A housing in the form of a container 140 for an agitator, such as impeller 118, is provided in a lower portion, such as within a base chamber 116.

As perhaps best understood with reference to the cross-sectional, enlarged view of FIG. 6A, the container 140 includes an upper portion 140a with a receptacle or fitment, which may comprise a tubular connector 140b, for receiving or connecting to a fluid (e.g. gas) injector. With combined reference to FIG. 6, the injector may comprise a first conduit in the form of a flexible or semi-flexible tube 142 extending within the central chamber 126 to a fitment or tubular connector 114a associated with the cover 114 and in communication with an external source of fluid (e.g., gas) (not shown, but see FIGS. 5A, 5B). The connector 114a may be located at any location radially outward from a central axis X of the container 140, about which the impeller 118 rotates. The connector 114a may optionally include or otherwise communicate with a perforated portion including a plurality of holes 115 at an interface with the interior compartment of the container 140 for generating bubbles for delivery into the path of travel for the impeller 118. The perforations or holes 115 may form part of the fitment, or may be provided in an adjacent wall of the container 140 through which the gas is passed into the interior compartment thereof and into the path of agitator or impeller 118. The sizing of the holes 115 allows for the bubbles entering the container 140 to be custom-sized, as desired for a particular application.

Thus, gas delivered via tube 142 is introduced into the container 140 as bubbles of a first size created by holes 115. These gas bubbles when introduced into liquid within the container 140 flow directly into the path of the impeller 118 when rotating. As referenced hereinabove, contact with the rotating impeller 118 causes shearing of the bubbles, which tends to reduce their size. As a result of the pumping action created, the associated liquid with the smaller-sized bubbles as a result of the shearing action created by engagement with the rotating impeller 118 is then ejected radially outwardly from the container 140 via openings 141 and flows with the associated liquid into (vertically through) the one or more fixed beds of the bioreactor 100.

As noted above, in the case of a structured fixed bed comprising layers spaced in a radially outward direction, the microbubbles are better able to pass into and through channels formed by the spacer layers (not shown in FIG. 6, but see FIG. 5) and the adjacent cell immobilization layers (or other available paths) of the fixed bed 122 (e.g., through pores in the woven substrate forming the fixed bed). This serves to further enhance the oxygenation of the cells being grown in the bed, without a corresponding need to increase the speed of the impeller 118 and the resulting liquid flow rate.

FIG. 6A further illustrates that a post 146 about which the impeller 118 rotates may extend at least partially within the container 140. This post 146 may be hollow, thus forming a tube extending to the base of the container 140 and, thus, bioreactor 100, and may be adapted to connect with a tube 148 within the central chamber 126 such as upon passing through the upper portion 140a of the container 140. This tube 148 may also extend to and connect with a second connector 114b of the cover 114 (see FIG. 6). This tube 148 may also be used to deliver fluid (liquid, gas, or both) to or from the container 140 via post 146. The distal end of the tube 148 within the container 140 may be spaced above the floor F of the bioreactor 100, such as by a spacer, which may take the form of a crenulated portion 146b. For example, the tube 148 may be used as a drain for recovering liquid from the container 140, and hence the bioreactor 100, by applying negative pressure to the tube 148 from an external source (not shown), such as via fitment or connector 114b. This drain set up will maximize the fluid (and thus target biomolecule) drained from the bioreactor 100. Similar functionality could also be applied to tube 142, if desired. Although both tubes are shown in FIGS. 6 and 6A, the bioreactor of the present disclosure can include only one of the tubes.

Turning now to FIG. 6B, certain details of the arrangement of the bioreactor 100 of this embodiment may be further understood. In the illustrated example, the post 146 may serve to connect with and support at least part of a bearing 149 for the impeller 118. Specifically, the bearing 149 may include a lower portion 148a forming a first race 148b for supporting rolling elements, such as balls 151. The impeller 118 may include the upper race 118a for capturing the balls 151 in an annular passage thus formed between the two races to provide low-frictional rotational movement for the impeller 118. A retainer 146a on the post 146 may serve to retain the impeller 118, and may comprise a snap-fit arrangement (not shown) for receiving and retaining impeller 118 in a releasable manner.

Referring to FIGS. 6C and 6D, the impeller 118 may also include a plurality of upstanding blades or vanes 118b. These vanes 118b may be connected to a base 118c (which incorporates one or more magnets 118d in the case of a non-contact drive arrangement). The vanes 118b may be curved in a radial direction (that is, the upstanding walls forming the vanes 118b may curve as they extend radially outwardly, with the degree of curvature varying in the circumferential direction, in the illustrated for clockwise movement). The vanes 118b may also change in height vertically from a higher portion near the center (H₁), adjacent to the rotational axis X, to a lower portion (H₂) adjacent a periphery of the impeller 118, as perhaps best understood from FIG. 6D.

As can be appreciated from FIG. 6A, the container 140 may comprise the upper portion 140a noted above, as well as a lower portion 140c, which may include an upstanding wall 152 with various openings 141 for transmitting liquid, such as in a radial direction to an outer chamber, eventually for passage through the cell culture bed. The upper portion 140a may include a lower wall 154 that slopes upwardly as it extends radially inwardly in order to accommodate the varying height vanes 118b of the impeller 118. This arrangement serves to minimize overhead space in the container 140 and ensure that bubbles are sucked into the container 140 (if introduced outside the container) and efficient shearing action to divide the bubbles introduced by tube 142 is achieved. The disclosed form of impeller 118 also serves to increase torque transmission to the liquid at a given rotational speed, as compared to a flat-bladed impeller.

The lower wall 154 may also connect to an upstanding wall 156 adapted to seal with the central column of the bioreactor, such as by snap fit engagement. Thus, liquid flow is urged into a central opening 159 in the wall 154 to enter the container 140. An anti-vortex structure, such as for example in the form of upstanding generally square panels 161 (e.g., four spaced 90 degrees apart about the axis X), may also be provided adjacent to the opening 159, and may form part of the lower wall 154.

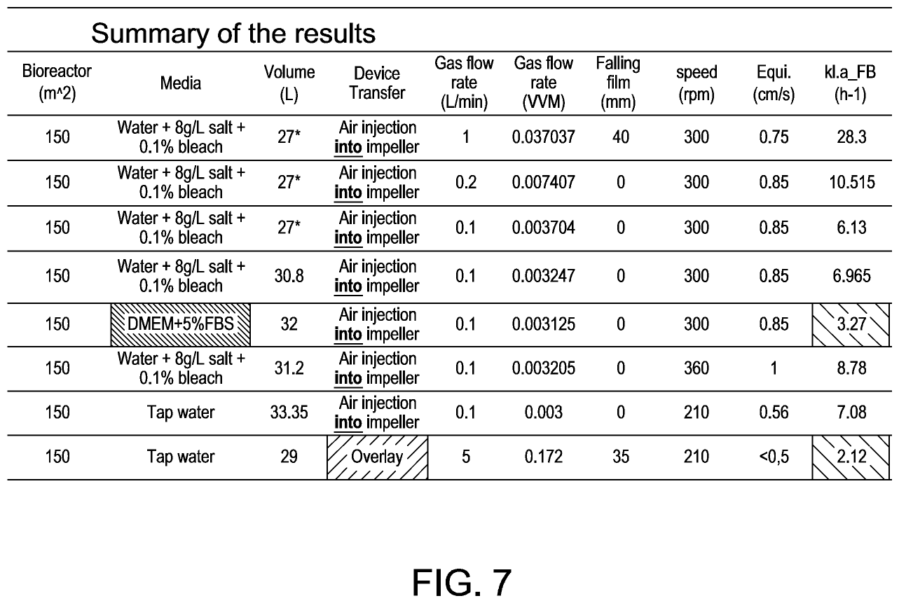

The table in FIG. 7 includes experimental data results for a fixed bed bioreactor with and without air injection into the impeller region. It can be understood from the data that, for a substantially lower gas flow rate (row 5), a higher kLa value was achieved as compared to a much higher gas flow rate using a conventional sparger not associated with the impeller (such as in row 8). It is clear that the introduction of the air into the fixed bed bioreactor as described above and shown in the corresponding figures consistently impacts the kLa in a positive manner without having the problems of such use in other bioreactors.

Additionally (or alternatively), increasing the gas transfer coefficient, or kLa, value may be achieved by increasing or extending the distance, and thus the time during which, the liquid media travels while in contact with gas phase prior to returning to the fixed bed. In one possible embodiment, this extended flow pattern is achieved by providing a flow extender (which for purposes of this disclosure means a structure with an increased surface area over which the liquid must flow in order to increase the residence time in contact with gas, and may be formed of any suitable material, such as polymer, metal, or the like). It may also be desirable, as discussed further below, to combine the flow extender with a flow disruptor to cause turbulence, which minimizes the diffusion layer normally formed at the gas/liquid border. One skilled in the art will appreciate that this introduction of a flow extender and/or flow disrupter can be applied to any fixed bed bioreactor design including an unstructured packed bed bioreactor.

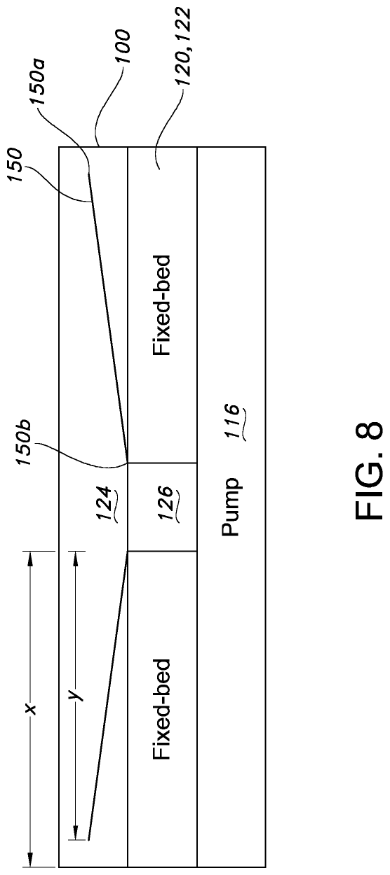

FIG. 8 shows a schematic representation of a bioreactor with a flow extender according to an embodiment of the disclosure. Flow extender 150 is illustrated in the form of an annular, frusto-conical plate-like structure located in the chamber 124 of the bioreactor 100, such zone including a headspace. In this particular arrangement, liquid exiting the top of the fixed bed 122 as a result of the action of the centrifugal pump is thus forced radially outwardly by the underside of the plate, and caused to flow over an outer peripheral edge 150a of the flow extender 150, and then along the opposite or upper surface of plate, toward a central opening forming a return or outlet at the inner edge 150b to return liquid to the upper end of the central chamber 126.

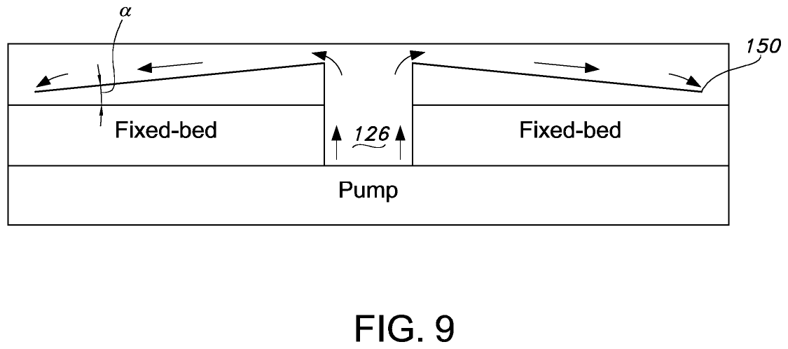

The flow extender 150 in this embodiment has a radial dimension Y. This radial dimension Y may be at least 50% of the radial dimension X of the annular chamber 120, but instead may be anywhere between 1-99% of X. In this manner, the liquid flow is diverted from a more direct path to entering the return (e.g., column forming chamber 126) such that the residence or contact time between the liquid and the headspace gas in the chamber 124 is increased. While the outer edge 150a is shown as being elevated relative to the inner edge 150b, it can be appreciated that the arrangement may be reversed, such as in a situation where the direction of liquid flow is radially outward (but the concept of creating a longer flow path remains the same), as shown in FIG. 9. The slope of the surface may be at an angle α relative to a horizontal plane of 1-85° and, specifically 1-45°.

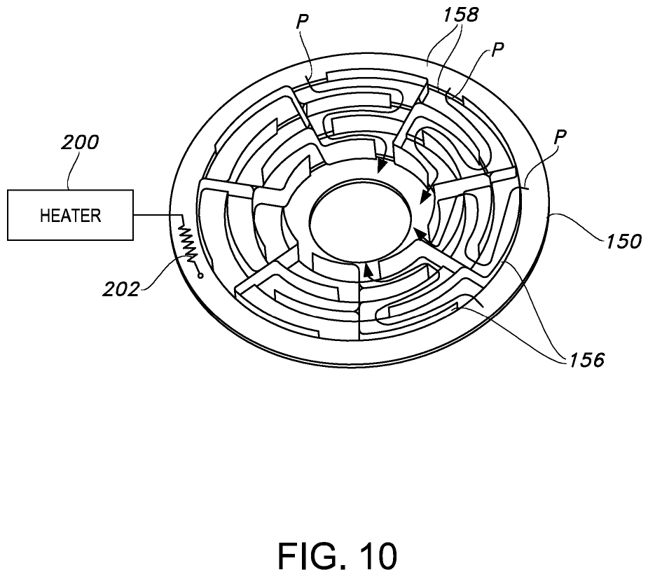

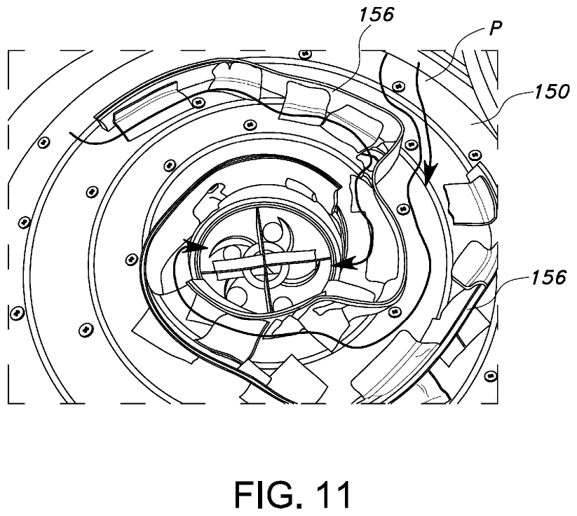

While the flow extender 150 shown in FIGS. 8 and 9 includes smooth, sloping liquid-engaging surfaces, modifications can be introduced to further enhance the residence time of the liquid in connection with the gas in the chamber 124. For example, as shown in FIG. 10, the flow extender may be provided with one or more winding or maze-like passages for further increasing the residence time of the liquid in moving from an exit point of the bed (e.g., spiral bed 122) to a location for returning for re-entry into the bed. These labyrinthian passages may be provided by upstanding walls 156 extending substantially radially and substantially circumferentially (note arrow P showing resulting path), and forming a liquid inlet at one end and a liquid outlet at the other end of the passage. The walls 156 may be regular (i.e. forming each passage with a substantially identical shape), as shown in FIG. 10, or irregular, as shown in FIG. 11. The extender may also be discontinuous in the circumferential direction, which may be advantageous if there is a desire to sample the underlying bed, such as spiral bed 122, using any gap in the path created to access the same.

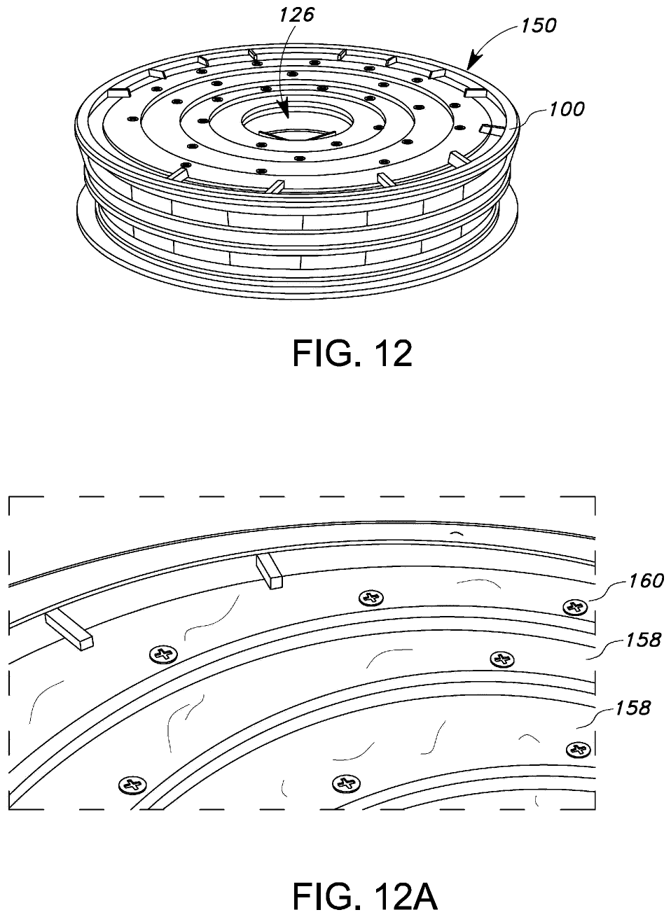

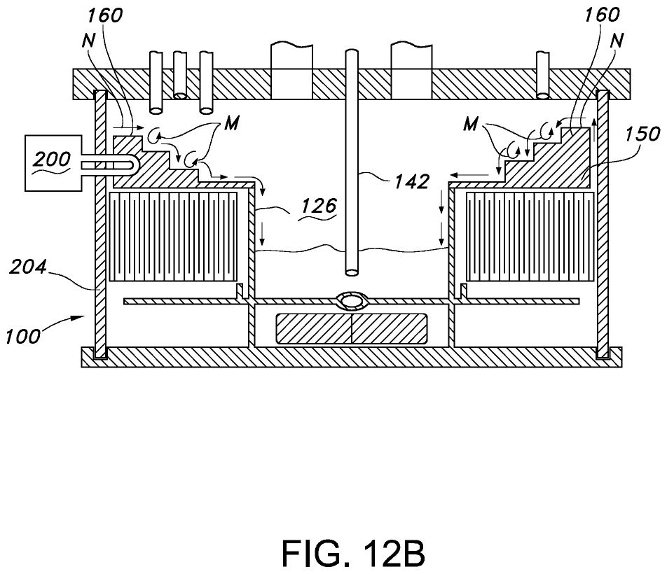

As further shown in FIG. 11, as well as in FIGS. 12, 12A, and 12B, the flow extender 150 may be provided in a modified form with a series of concentric ledges, or steps 158, over which liquid flows or cascades while moving radially. This not only increases the residence time of the liquid, but also generates a measure of turbulence as the liquid changes direction while flowing along and/or over the steps 158. Specifically, as can be appreciated from FIGS. 12A and 12B, the flow over the first generally planar tread 160 of the flow extender 150 is laminar (arrows N), but then becomes increasingly turbulent (arrows M) as it moves along and over the steps 158, descending toward the point of return to the bed (such as via central chamber 126). This improves the oxygenation of the liquid prior to re-entering the bed. The height and radial extent of the steps 158 may be the same or varied, and any number of steps may be provided depending on the particular application (also note the possible inclusion of tube 142 per the above-described embodiment).

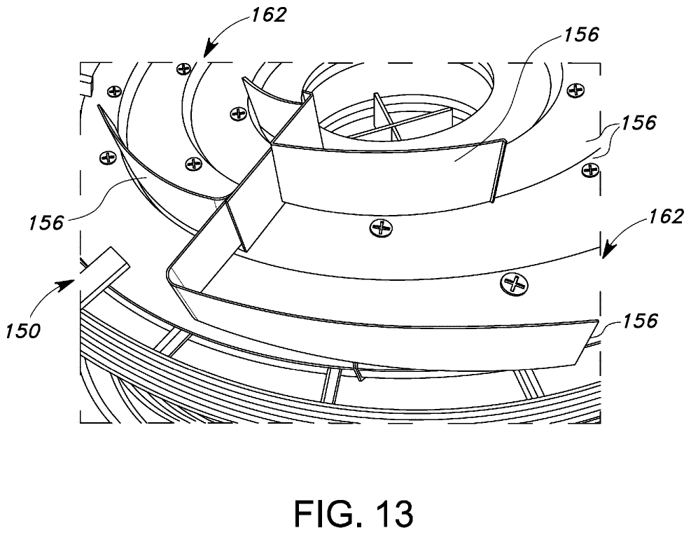

FIG. 13 illustrates that walls 156 may be used in combination with the steps 158, and, optionally, in an irregular manner, to create pockets 162 at selected locations on the extender 150. These pockets 162 further enhance the residence time of the liquid after exiting the bed 122 and prior to re-entry. As can be appreciated, the radial extent, circumferential extent, and number of pockets 162 may vary depending on the application, and strategic placement of these flow extender mechanisms may be accomplished to increase the liquid residence time in order to achieve optimal gas (oxygen) transfer.

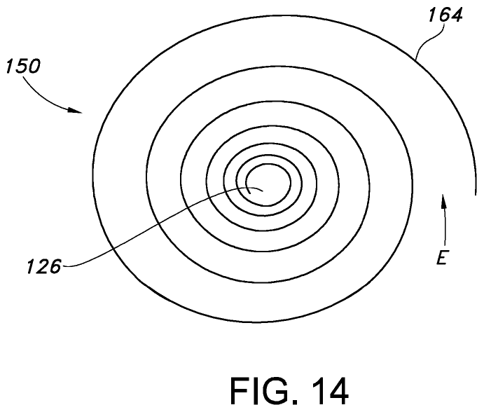

FIG. 14 illustrates a further embodiment in which the extender 150 may include a surface with a spiral wall 164 upstanding therefrom. The spiral wall 164 has one or more entrances E for admitting liquid. The liquid may then flow along the spiral path created prior to entering the central chamber 126 (or paths radially outside the fixed bed in some designs), which thus increases the opportunity for oxygen transfer. As with the other embodiments, the arrangement could be reversed, such that the entrance to the spiral path created by the wall 164 for liquid exiting the bed is radially inward of the location for returning liquid to the bed.

In any of the foregoing embodiments, the extender 150 may also be provided with appendages or surface texture to create turbulence in the liquid as it traverses across the corresponding surface. Such turbulence caused thereby increases the opportunity for gas transfer to the liquid media. For example, embosses (e.g., circumferential grooves, radial grooves, or a combination thereof) or bosses (e.g., circumferential projections, radial projections, or a combination thereof) may be provided in the surface for causing deviations in the flow of liquid along the plate.

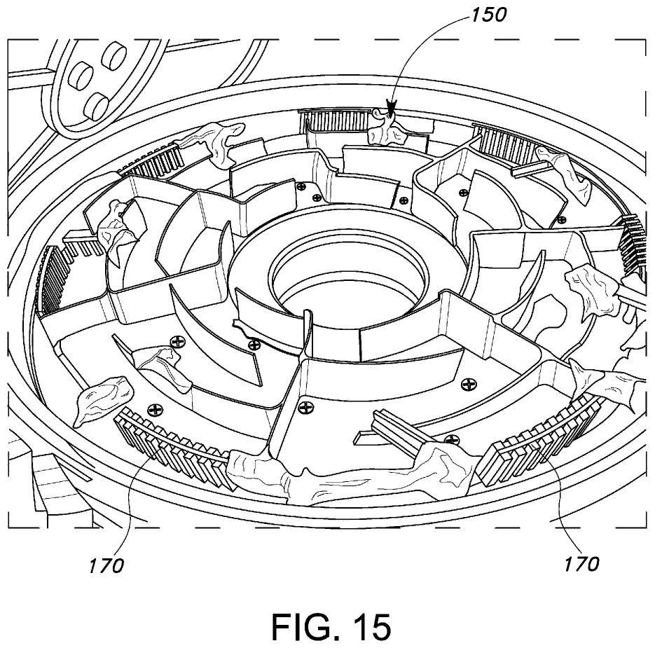

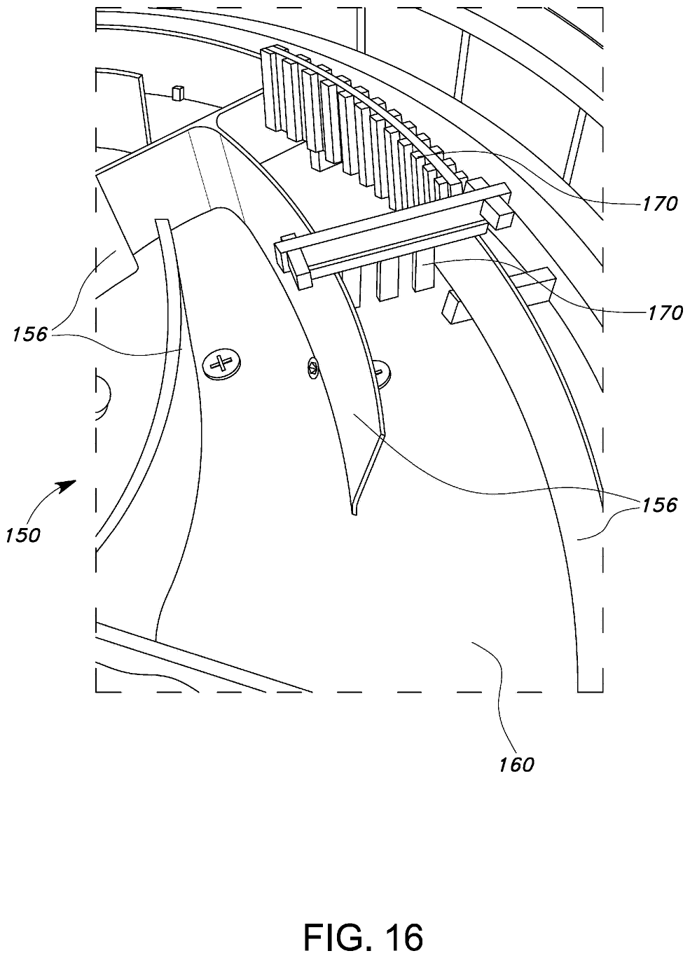

Likewise, the extender may also include with forks, posts, pins, barriers or like structures to create turbulence in the flow. For example, as shown in FIGS. 15 and 16, flow disrupting gates 170 having one or more posts (shown as vertical, but could be angled or horizontal) may be located on or adjacent to the extender 150 and/or in the flow path created along it to provide further disruption to the liquid flow.

One skilled in the art will appreciate that the gates 170 can be formed of vertical posts as shown but could also be formed by angled or horizontal posts or a combination of any gate- or fence-like structure. The gate(s) 170 thus serve to create further disruptions in the liquid flow, thus increasing the turbulence and improving the gas transfer. As perhaps best shown in FIG. 16, the gates 170 may be elongated either or both of a circumferential direction and a radial direction (with the circumferentially extending gate along the periphery of extender 150 serving to cause turbulence in liquid at the entrance to the extender, while the radially extending gate causes turbulence in the liquid flowing circumferentially along the extender, such as along the generally planar tread 160 thereof and between upstanding walls 156).

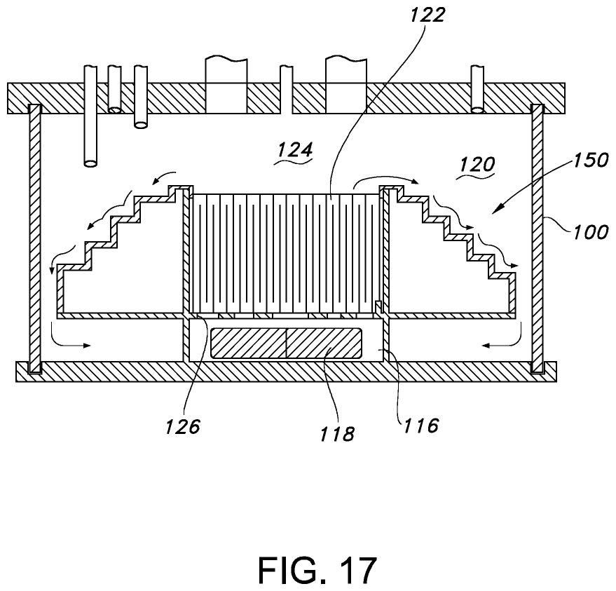

While the above embodiments illustrate the flow extender in association with the zone or chamber 124 including a headspace in the bioreactor 100, it can be appreciated that the extender concept may be applied to other locations in the bioreactor. For instance, as shown in FIG. 17, the extender 150 may be provided in a radially outer chamber 120 and the bed 122 may be located in the radially inward (central) chamber 126. The impeller 118 may be in a base chamber 116, and serves to cause liquid to flow upwardly through the bed 122, along the extender 150 to interact with the gas in chambers 120, 124, and then back to the base chamber. In the illustrated example, the extender 150 comprises steps descending in a radially outward direction, but this is just an example and other forms of extenders could be used where there is a gas phase and liquid phase interaction. Also, while this stepped extender 150 is shown as a solid structure in cross-section, it could also take the form of a staircase with the flow able to contact the underside thereof.

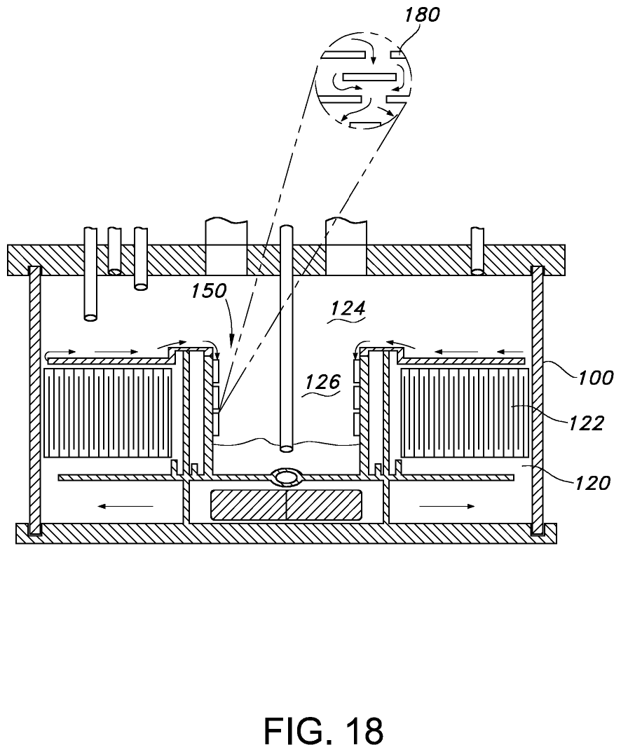

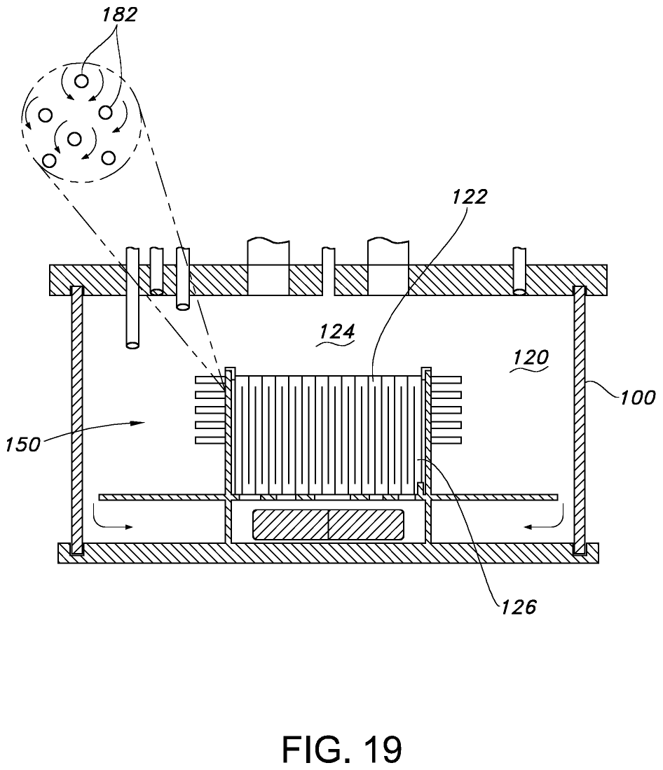

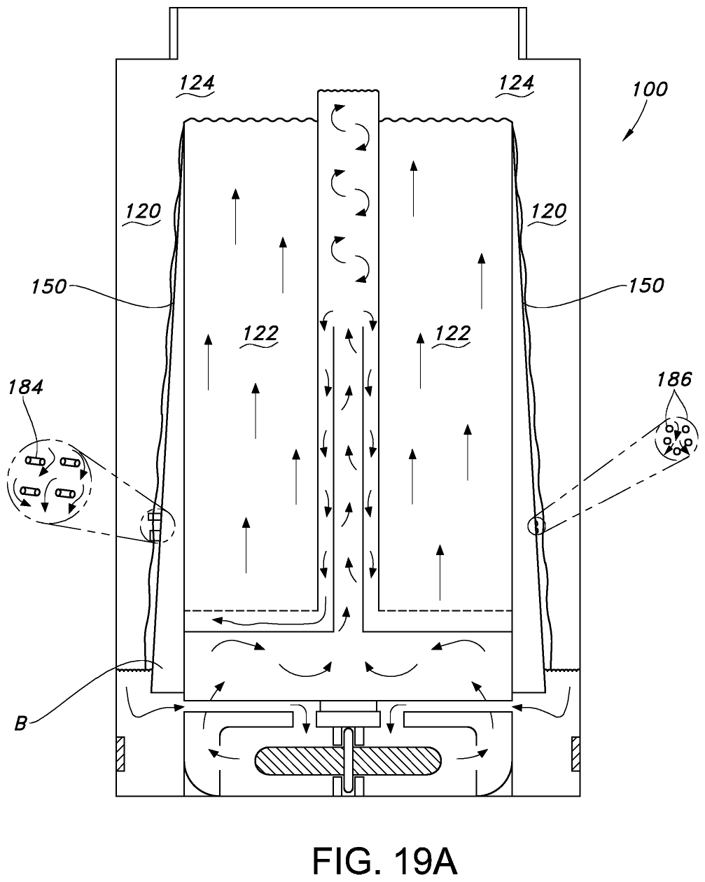

In a case where the bed 122 is in the outer (annular) chamber 120, the extender 150 also may be located within the central chamber 126, as shown in FIG. 18. In this example, the extender 150 comprises a maze-like structure formed of segmented walls 180 that may be spaced vertically and circumferentially to create a tortuous flow in a film of liquid exiting the bed 122 and entering chamber 126 from above. This creates the desired disruption of the normally occurring diffusion layer and thus causing the liquid to interface with the gas in chambers 124, 126, thereby improving the gas transfer. The structure of the extender 150 for causing the flow disruption could also take the form of pins, forks, or the like. As shown in FIG. 19, the extender 150, such as for example one comprising pins 182 or any other disruptive structures, may also be provided in chamber 120, when the bed 122 is located in central chamber 126.

Turning back to FIG. 6, it can be understood that the flow extender(s) 150 may be formed by an annular, inwardly directed lip or projection 181 along the inner surface of the columnar central chamber 126. Each projection 181 may be angled or chamfered to aid in disrupting liquid flow travelling vertically along the inner wall of this chamber 126, thereby improving the gas transfer. Specifically, this creates the desired disruption of the normally occurring diffusion layer and thus causes the liquid to interface with the gas in chamber 126. Each projection(s) 181 may form part of a stackable, tubular structure 101 for interconnecting so as to form the central chamber 126, and also provides the inner wall of the radially outward chamber(s) 120 for receiving each layer of fixed bed 122 present.

FIG. 19A illustrates a further example, in which a bioreactor 100 includes a centralized bed 122 through which liquid is caused to flow upwardly into chamber 124 forming headspace, and then downwardly along a gently sloping surface so as to maximize the residence time of the liquid in the headspace. The slope of the surface may be at an angle relative to a horizontal plane of 1-85° and, specifically 1-45°. The flow extender 150 may further be provided with turbulence-causing structures, such as surface protrusions 184, which may be in the form of bumps provided on the liquid-contacting surface of the extender 150. This creates the desired disruption of the normally occurring diffusion layer and thus causes the liquid to interface with the gas in chamber 120, thereby improving the gas transfer. The extender 150 in this version could also be stepped, or could have any other form of turbulence-generating structures described herein or otherwise.

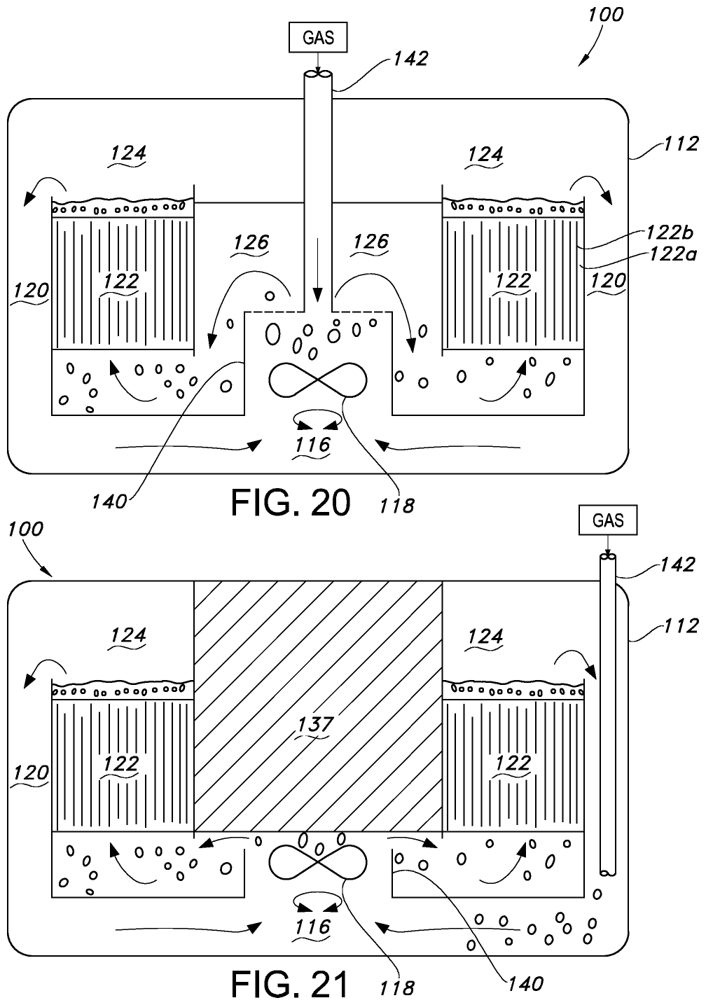

Turning to FIG. 20, an alternative embodiment of a bioreactor 100 is illustrated, which as above comprises a housing 112. In this embodiment, liquid is circulated by an agitator such as an impeller 118 in a housing or container 140 in or near the base chamber 116 and flows to a central chamber 126 first, then radially outwardly to pass vertically through a structured fixed bed 122 (such as the version shown in FIGS. 3 and 3A-3C). The liquid upon exiting the upper portion of the bed 122 then returns along a chamber 120 radially outward of the bed 122, and is returned to the impeller 118 for repeating the cycle.

As shown, this arrangement may benefit from the implementation of various optional features described herein. For example, as shown, a gas may be introduced into the bioreactor 100 by tube 142, such as directly to the container 140 for impeller 118 or to one or more chambers 116, 120, 126 by re-positioning the outlet of the tube 142 and/or providing multiple tubes. In any case, the action of the impeller 118 serves to create shear and divide any gas bubbles into microbubbles, which then enter the bed 112 as a result of the flow pattern created. Given their smaller size, the microbubbles are better able to pass into and through channels formed by the spacer layers 122b and the adjacent cell immobilization layers 122a (or other available paths) of the fixed bed 122. This serves to further enhance the oxygenation of the cells being grown in the bed, without a corresponding need to increase the speed of the impeller 118 and the resulting liquid flow rate.

Indeed, as the microbubbles pass through channels formed by the spacer layers 122b and the adjacent cell immobilization layers 122a (or other available paths in case spacer layer is omitted), they tend to break up into yet smaller microbubbles as they travel therethrough. Hence, the gas transfer during residence time in the fixed bed is increased and the bubbles emanating from the bed 122 once having passed therethrough are actually still even smaller in size that at a point of entry, as a result of shearing forces caused by the bubbles engaging the mesh of the spacer layer 122b in the illustrated embodiment. Moreover, the release of the gas into or near the agitator container 140 and the resulting flow avoids the creation of deleterious air pockets in the bioreactor 100, which are notoriously difficult to remove without halting the bioreactor operation.

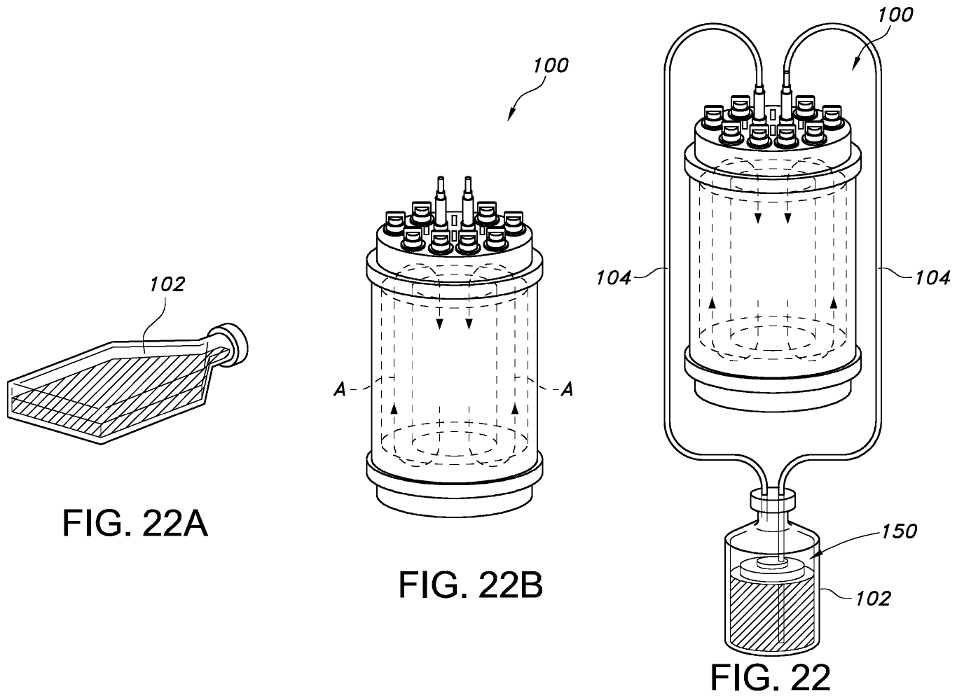

FIG. 21 illustrates an alternative embodiment of a bioreactor 100 somewhat similar in construction to that of FIG. 20, with the main exception that the central chamber is omitted takes the form of a solid core 137. Thus, liquid may exit the container 140 for impeller 118 radially through openings, and travel essentially as previously described, returning to the container 140 via base chamber 116. In view of the solid nature of the columnar central chamber 126, the gas introduction, if desired, may be achieved by introducing a tube 142 into the outer chamber 120 along one or more sides of the bioreactor 100, with the “macrobubbles” thereby being introduced into the container 140, being sheared or divided by impeller 118, and then introduced to bed 122 (which again may create further reductions if in the form described herein).

The embodiments of bioreactors 100 shown in FIGS. 20-21 may also benefit from the inclusion of the flow extender (not shown). For example, the flow extender may be located in the chamber 124 forming headspace, or in the outwardly directed chamber 120, to further increase the residence time of the liquid in connection with any gas therein. One skilled in the art can appreciate that the flow extender in chamber 124 could include a flow diverter of liquid exiting the bed 122 that acts to divert the flow toward the inside portion of the bed and then travel along such flow extender toward the outside return chamber 120 (which is opposite to the flow show in, for example, FIG. 18). Structures (as described above in other embodiments herein) for increasing the turbulence of the liquid flow may also be provided.

As can be appreciated, the flow extender 150 may be integrally formed as part of the bioreactor 100, or may be provided as an optional insert that may be added or removed as necessary or desired. The flow extender 150 is also readily scalable for use in a wide variety and sizing of bioreactors, including other forms not shown in the exemplary drawing figures.

As indicated in FIGS. 22, 22A, and 22B, in some embodiments, the bioreactor 100 may be used in connection with an external reservoir 102 and conduits 104 (e.g., forward and return) to form a continuous loop for circulating liquid to the bioreactor 100. As indicated in FIG. 22, the flow extender 150 may be located on the external reservoir 102, rather than in the bioreactor itself 100. Likewise, in the case where the reservoir 102 includes a pump for circulating liquid, it could include a tube (not shown) for delivering gas bubbles, which are then divided by the agitator in the bioreactor 100 prior to delivery to the bed.

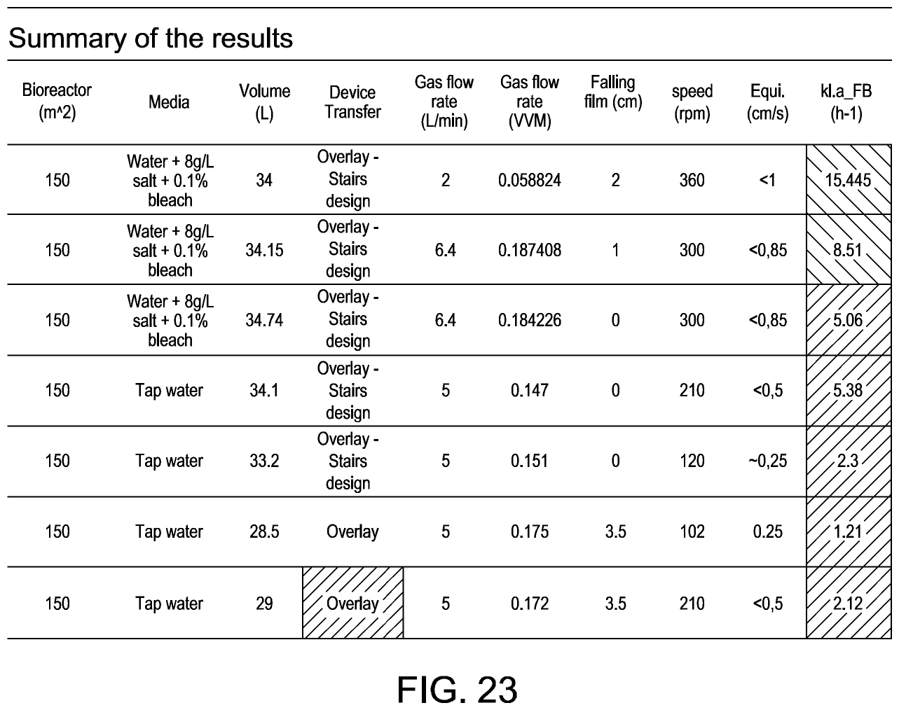

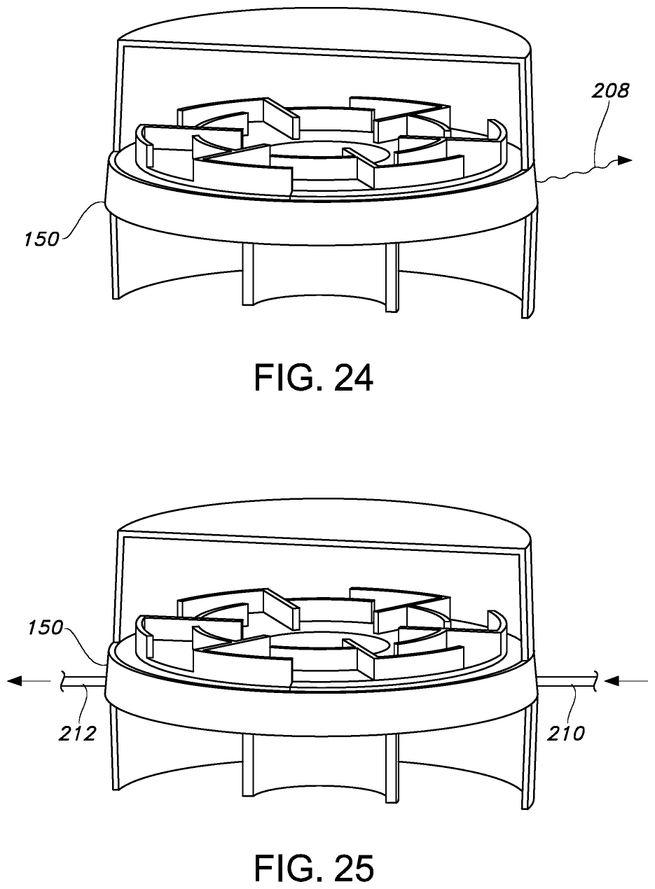

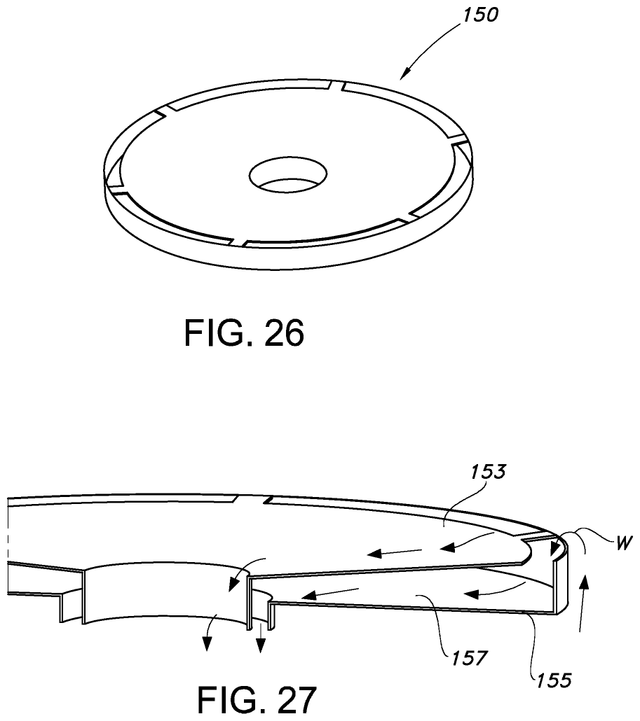

The table of FIG. 23 illustrates the improvement in kLa with use of one such embodiment of extender having steps (rows 1-5), as compared to with no such extender (rows 6-7). Comparing rows 4 and 7 in particular, it can be appreciated that a substantial increase in the kLa value results from the use of the extender, as compared to without, at the same impeller speed.

A further aspect of the disclosure pertains to thermal regulation of the bioreactor and, in particular, thermal regulation of the liquid encountering the cells, which may be achieved using a thermoregulator for regulating the temperature of the liquid within the interior of the bioreactor itself. For example, as shown in FIG. 10, a thermoregulator, such as a heater 200, may be provided for increasing the temperature of the flow extender 150.

The heater 200 itself may be located partially external to the bioreactor. The heater 200 may be, for example, a resistive heater wherein electrical current is supplied to a resistive element 202 associated with the extender 150, such as along a base thereof including one or more of the steps 158, one or more of the upstanding walls 156, or both. Alternatively, the heater 200 may comprise a radiator, which may comprise a heating element for warming a liquid external to the bioreactor, which is then delivered to the extender 150 (such as via a recirculation loop 204, as shown in FIG. 12B) to warm it. The extender 150 may comprise a material that is an efficient thermal conductor, such as for example a metal or polymer. Instead of a heater 200, the thermoregulator may also comprise a chiller for cooling the bioreactor.

Further examples are illustrated in FIGS. 24 and 25. In FIG. 24, the flow extender 150 is associated with a heater, which may take the form of an electric wire 208 for heating the flow extender (but the heating could also be applied to a separate part adjacent to or in contact with the flow extender). Alternatively, or additionally, as shown in FIG. 25, the flow extender 150 may be provided with an inlet 210 for receiving a thermally regulated liquid into an interior chamber or passage (not shown), and an outlet 212 for releasing the same.

In any case, when the liquid contacts the extender 150 and contacts the thermally regulated surfaces (floor and/or walls, which may naturally be heated if in thermal communication with the floor of extender), the temperature of the liquid is regulated (which temperature may be monitored via feedback loop using one or more sensors to ensure adequate control). Of course, the extender 150 functions to increase the residence time of the liquid and/or create turbulent flow, as described previously, and thus helps to ensure that the liquid temperature regulation is reliably achieved as a result of the contact, even if the velocity of the liquid is relatively high (which also helps to prevent overregulating). The extender 150 also has a large surface area, and thus helps to maximize the amount of contact with the liquid and the resulting heat transfer that may be achieved, which may reduce the amount of energy input (especially compared to a film applied to an external wall of a bioreactor). Using the proposed approach, thermal regulation internal to the bioreactor may be achieved in a manner that does not rely on thermal transfer through external walls, since direct contact between the heating element and liquid is achieved. This ensures that the temperature of the liquid may be regulated more efficiently and avoids damage to cells fixed in the bed.

Reference is also made to FIGS. 26-27, which illustrate a flow extender 150 in the form of an open shell, which allows for liquid to flow both along an upper surface 153 (which may be sloped) and a lower surface 155, which is shown as being generally planar but could also be sloped. When used in connection with the bioreactor of FIGS. 1 and 2, it can be understood that liquid would flow vertically from outer chamber (not shown) and over the periphery (as indicated by arrow W), at which point a portion could enter a chamber 157 between the surfaces 153, 155, while another portion would travel over the upper surface 153 (assuming a sufficient volume of fluid flow).

As can be appreciated, this increases the total surface area for contacting the flowing liquid, which may enhance the thermoregulation in the case where the surfaces are heated or cooled (which may be done in the ways previously described). The liquid flows may then recombine along the inner periphery, such as for being returned along the central column (not shown) One or both of the surfaces 153, 155 may also be provided with turbulence generating structures. It can also be appreciated that valves may be provided to regulate the amount of liquid entering the chamber 150c, which would thus form a regulator that could be used to control the thermal regulation of the liquid by controlling the amount in contact with the thermally regulated lower surface 150b. It can be appreciated that the liquid flow may also go in the opposite direction (e.g., upward from the central column, radially outwardly, and returning via the outer chamber).

This disclosure may be considered to relate to the following items:

1. An apparatus for culturing cells, comprising:
a bioreactor including a fixed cell culture bed;
an agitator for pumping liquid through the cell culture bed;
a container for the agitator; and
a first conduit connected to the container.

2. The apparatus of item 1, wherein the first conduit comprises an injector conduit for delivering gas bubbles into the container.

3. The apparatus of item 1, wherein the first conduit comprises a drain conduit for draining liquid from the bioreactor.

4. The apparatus of item 2, further including a second conduit comprising a drain conduit for draining liquid from the bioreactor.

5. The apparatus of any of items 1-4, wherein the bioreactor comprises a central chamber and a cover, the first conduit extending from the cover and through the central chamber to an opening of the container.

6. The apparatus of any of items 1-5, further including a tubular post associated with the container, the first conduit being connected to the tubular post.

7. The apparatus of any of items 1-6, wherein the agitator comprises a, preferably magnetic, impeller rotatably coupled to the tubular post by a bearing.

8. The apparatus of any of items 1-7, wherein the agitator comprises a plurality of curved vanes.

9. The apparatus of any of items 1-8, wherein the agitator comprises a plurality of vanes, each vane of the plurality of vanes having a higher central portion and a lower peripheral portion so as to create a variable height.

10. The apparatus of item 9, wherein the container comprises a sloped upper portion adapted to accommodate the variable height agitator.

11. The apparatus of any of items 1-10, wherein the container includes a fitment adapted to form custom-sized bubbles from a gas delivered thereto by the first conduit.

12. The apparatus of item 11, wherein the fitment comprises a perforated portion.

13. The apparatus of any of items 1-12, wherein the cell culture bed is adapted to divide/shear any gas bubbles in the liquid.

14. The apparatus of any of items 1-13, wherein the cell culture bed comprises a fixed bed.

15. The apparatus of any of items 1-14, wherein the fixed bed comprises a structured fixed bed.

16. The apparatus of any of items 1-16, wherein the structured fixed bed comprises alternating layers comprising one or more cell immobilization layers, and optionally one or more spacer layers.

17. The apparatus of item 17, wherein the one or more cell immobilization layers or the one or more spacer layers form paths to divide or shear gas bubbles in the liquid that travel through the fixed bed.

18. The apparatus of any of items 1-17, wherein the cell culture bed is located in an annular outer chamber of the bioreactor adapted to receive a flow of liquid from a pumping action created by rotation of the agitator in the container.

19. The apparatus of any of items 1-18, further including a plurality of cell culture beds arranged in a stacked configuration within an annular outer chamber of the bioreactor.

20. The apparatus of any of items 1-19, wherein the bioreactor further includes a flow extender, possibly with a thermoregulator.

21. An apparatus for culturing cells, comprising:
a bioreactor including a cell culture bed;
an agitator for pumping liquid through the cell culture bed; and
an injector for injecting a gas into the liquid to form first bubbles having a first size, the agitator converting the first bubbles to second bubbles having a second, smaller size than the first size for delivery to the cell culture bed with the liquid.

22. The apparatus of item 21, wherein the agitator is located in a container in fluid communication with the bioreactor, the container including at least one inlet and at least one outlet.

23. The apparatus of item 21 or item 22, wherein the injector comprises a tube having an open end located upstream of the at least one inlet to the container.

24. The apparatus of any of items 21-23, further including a housing having a central chamber associated with the at least one inlet and located radially inward of the cell culture bed, wherein the open end of the tube is positioned within the central chamber.

25. The apparatus of any of items 21-24, wherein the injector comprises a tube having an open end located in a fluid flow path between the at least one inlet and the at least one outlet.

26. The apparatus of any of items 21-25, wherein the cell culture bed comprises a fixed bed.

27. The apparatus of any of items 21-26, wherein the fixed bed comprises a structured fixed bed.

28. The apparatus of any of items 21-27, wherein the structured fixed bed comprises alternating layers comprising one or more cell immobilization layers and one or more spacer layers.

29. The apparatus of item 28, wherein a channel is formed between the one or more cell immobilization layers and one or more spacer layers to divide/shear any gas bubbles in the liquid into a third bubbles having a third yet smaller size than the second size.

30. An apparatus for culturing cells, comprising:
a bioreactor including a cell culture bed;
means for pumping liquid through the cell culture bed; and
means for injecting a gas into the liquid to form first bubbles having a first size, the pumping means converting the first bubbles to second bubbles having a second, smaller size than the first size for delivery to the bed with the liquid.

31. The apparatus of item 30, wherein the means for pumping comprises an agitator.

32. The apparatus of item 30 or item 31, wherein the means for injecting the gas comprises a tube connected to a gas supply.

33. A method of culturing cells in a cell culture bed, comprising:
providing a liquid with gas bubbles;
agitating the liquid to divide the gas bubbles; and
delivering the liquid with the divided gas bubbles to the cell culture bed.

34. The method of item 33, wherein the providing step comprises supplying a gas to the liquid upstream of an agitator for performing the agitating step.

35. The method of item 33 or item 34, wherein the providing step comprises supplying a gas to the liquid to a container including an agitator for performing the agitating step.

36. The method of any of items 33-35, wherein the cell culture bed comprises alternating layers comprising one or more cell immobilization layers and one or more spacer layers forming a channel for the liquid through the bed, the layers performing the step of further dividing the bubbles as the liquid passes through the cell culture bed.

37. A method for culturing cells, comprising:
a bioreactor including a cell culture bed;
an agitator for pumping liquid through the cell culture bed; and
an injector for injecting a gas into the liquid to form bubbles having a first size;
wherein the agitator is adapted to divide or shear the gas bubbles into a second size smaller than the first size;
wherein the cell culture bed is adapted to divide or shear the gas bubbles into a third size smaller than the second size.

38. An apparatus for culturing cells, comprising:
providing a liquid with gas bubbles having a first size;
agitating the liquid to divide the gas bubbles to a second size smaller than the first size; and
delivering the liquid with the divided gas bubbles to the cell culture bed, which divides the bubbles to a third size smaller than the second size.

39. An apparatus for culturing cells, comprising:
a housing for containing a liquid for delivery to a bed for culturing cells, the housing including a zone having a gas; and
a flow extender adapted for extending a residence time and causing turbulence in the liquid in the zone.

40. The apparatus of item 39, wherein the flow extender comprises a frusto-conical structure having surface structures for causing the turbulence.

41. The apparatus of item 39 or item 40, wherein the flow extender comprises one or more labyrinthian, maze-like or winding passages.

42. The apparatus of any of items 39-41, wherein the flow extender comprises a plurality of steps.

43. The apparatus of any of items 39-42, wherein the housing is arranged with an outer chamber for receiving the bed, the chamber being an upper chamber including a headspace being located above a liquid exit of the bed, and further including a central chamber for returning liquid for entering the bed.

44. The apparatus of any of items 39-43, wherein the housing is arranged with an outer chamber for receiving the bed, the chamber being a central chamber for returning liquid for entering the bed.

45. The apparatus of any of items 39-44, wherein the housing is arranged with an inner chamber for receiving the bed, the chamber being an outer chamber for returning liquid for entering the bed.

46. The apparatus of any of items 39-45, wherein the flow extender has a peripheral edge greater in height relative to a radially inward edge of the extender.

47. The apparatus of any of items 39-46, wherein the housing comprises an annular chamber including the bed and having a radial dimension X, and wherein a radial dimension Y of the extender is greater than 0.5X.

48. The apparatus of any of items 39-47, wherein the flow extender is capable of being heated.

49. An apparatus for culturing cells, comprising:
a housing for containing a liquid for delivery to a bed for culturing cells, the housing including a headspace having a gas; and
a flow extender adapted to extend a residence time in the headspace.

50. The apparatus of item 49, wherein the flow extender comprises a frusto-conical plate.

51. The apparatus of item 49 or item 50, wherein the flow extender comprises one or more maze-like or winding passages.

52. The apparatus of any of items 49-51, wherein the flow extender comprises a plurality of steps.

53. The apparatus of any of items 49-52, wherein the housing is arranged with an outer chamber for receiving the bed, the headspace being located above a liquid exit of the bed, and further including a central chamber for returning liquid for entering the bed.

54. The apparatus of any of items 49-53, wherein the flow extender has a peripheral edge greater in height relative to a radially inward edge of the extender.

55. The apparatus of any of items 49-54, wherein the housing comprises an annular chamber including the bed and having a radial dimension X, and wherein a radial dimension Y of the extender is greater than 0.5X.

56. The apparatus of any of items 49-55, wherein the flow extender is heated.

57. An apparatus for culturing cells, comprising:
a housing for containing a liquid for delivery to a bed for culturing cells, the housing including a centrifugal pump for pumping liquid through the bed and into a headspace;
a flow extender adapted for increasing a residence time of the liquid in the headspace; and
an injector for injecting a gas to the liquid to form first bubbles having a first size, the centrifugal pump converting the first bubbles to second bubbles having a second, smaller size than the first size for delivery to the bed with the liquid being pumped.

58. The apparatus of item 57, wherein the flow extender is heated.

59. An apparatus for culturing cells, comprising:
a housing for containing a liquid for delivery to a bed for culturing cells; and
a thermoregulator located within the housing for regulating a temperature of the liquid without directly heating or cooling the bed.

60. The apparatus of item 59, wherein the thermoregulator comprises a stepped structure.

61. The apparatus of item 59 or item 60, further including a structure having upstanding walls thermally regulated by the thermoregulator.

62. The apparatus of any of items 59-61, wherein the thermoregulator is located in a headspace of the housing.

63. The apparatus of any of items 59-62, wherein the thermoregulator comprises a power source external to the housing and a resistive element internal to the housing.

64. The apparatus of any of items 59-63, wherein the thermoregulator comprises a radiator.

65. An apparatus for culturing cells, comprising:
a housing for containing a liquid for delivery to a bed for culturing cells, the housing including a headspace having a gas; and
a flow extender adapted for extending a residence time and/or creating turbulence in the liquid in the headspace.

66. The apparatus of item 65, wherein the flow extender comprises a surface having a slope of 1-85° relative to a horizontal plane.

67. An apparatus for culturing cells, comprising:
a housing for containing a liquid for delivery to a bed for culturing cells, the housing including a zone having a gas; and
a flow extender adapted for extending a residence time of the liquid in the zone wherein the flow extender comprises a surface having a slope of 1-85° relative to a horizontal plane.

68. The apparatus of item 67, wherein the zone comprises a headspace.

69. An apparatus for culturing cells, comprising:
a housing for containing a liquid for delivery to a bed for culturing cells, the housing including a zone having a gas; and
a flow extender adapted for extending a residence time and/or causing turbulence in the liquid in the zone, the flow extender comprising an open shell.

70. The apparatus of item 69, wherein the open shell comprises a chamber for receiving a portion of the liquid.

71. The apparatus of item 69 or item 70, further including a regulator for regulating flow into the chamber.

72. The apparatus of any of items 69-71, wherein the open shell comprises a sloped upper surface.

73. The apparatus of any of items 69-72, further including a thermoregulator connected to the open shell.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” “generally” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows, e.g., “component includes” does not exclude or preclude the presence of additional, non-recited components, features, element, members, steps, known in the art or disclosed therein.

While preferred embodiments have been shown and described herein, it will be obvious to those skilled in the art that such embodiments are provided by way of example only. Numerous variations, changes, and substitutions will now occur to those skilled in the art without departing from the invention. For example, while the bioreactor is shown in a vertical orientation, it could be used in any orientation. The bioreactor may also be formed of rigid, flexible, or semi-flexible materials, and may be made for single or multiple uses. It should be understood that various alternatives to the embodiments of the invention described herein may be employed in practicing the invention. It is intended that the following claims define the scope of the protection under the applicable law and that methods and structures within the scope of these claims and their equivalents be covered thereby.

1. An apparatus for culturing cells, comprising:
a bioreactor including a fixed cell culture bed;
an agitator for pumping liquid through the cell culture bed;
a container for the agitator; and
a first conduit connected to the container.

2. The apparatus of claim 1, wherein the first conduit comprises an injector conduit for delivering gas bubbles into the container.

3. The apparatus of claim 1, wherein the first conduit comprises a drain conduit for draining liquid from the bioreactor.

4. The apparatus of claim 2, further including a second conduit comprising a drain conduit for draining liquid from the bioreactor.

5. The apparatus of claim 1, wherein the bioreactor comprises a central chamber and a cover, the first conduit extending from the cover and through the central chamber to an opening of the container.

6. The apparatus of claim 1, further including a tubular post associated with the container, the first conduit being connected to the tubular post.

7. The apparatus of claim 6, wherein the agitator comprises a, preferably magnetic, impeller rotatably coupled to the tubular post by a bearing.

8. The apparatus of claim 1, wherein the agitator comprises a plurality of curved vanes.

9. The apparatus of claim 1, wherein the agitator comprises a plurality of vanes, each vane of the plurality of vanes having a higher central portion and a lower peripheral portion so as to create a variable height.

10. The apparatus of claim 9, wherein the container comprises a sloped upper portion adapted to accommodate the variable height agitator.

11. The apparatus of claim 1, wherein the container includes a fitment adapted to form custom-sized bubbles from a gas delivered thereto by the first conduit.

12. The apparatus of claim 11, wherein the fitment comprises a perforated portion.

13. The apparatus of claim 1, wherein the cell culture bed is adapted to divide/shear any gas bubbles in the liquid.

14. The apparatus of claim 1, wherein the cell culture bed comprises a fixed bed.

15. The apparatus of claim 14, wherein the fixed bed comprises a structured fixed bed.

16. The apparatus of claim 15, wherein the structured fixed bed comprises alternating layers comprising one or more cell immobilization layers, and optionally one or more spacer layers.

17. The apparatus of claim 16, wherein the one or more cell immobilization layers or the one or more spacer layers form paths to divide or shear gas bubbles in the liquid that travel through the fixed bed.

18. The apparatus of claim 1, wherein the cell culture bed is located in an annular outer chamber of the bioreactor adapted to receive a flow of liquid from a pumping action created by rotation of the agitator in the container.

19. The apparatus of claim 1, further including a plurality of cell culture beds arranged in a stacked configuration within an annular outer chamber of the bioreactor.

20. The apparatus of any of claim 1, wherein the bioreactor further includes a flow extender, possibly with a thermoregulator.

21-73. (canceled)
